(19) United States
(12) Patent Application Publication
(10) Pub. No.: US 2003/0212095 A1
(43) Pub. Date: Nov. 13, 2003

(54) New bispidine compounds and their use in the treatment of cardiac arrhythmias

(76) Inventors: Kjell Andersson, Sodertalje (SE); Annika Bjore, Molndal (SE); Magnus Bjorsne, Molndal (SE); Fritiof Ponten, Molndal (SE); Gert Strandlund, Molndal (SE); Peder Svensson, Goteborg (SE); Louise Tottie, Molndal (SE)

Correspondence Address: Ropes & Gray, One International Place, Boston, MA, 02110-2624, US

(21) Appl. No.: 10/332,103

(22) PCT Filed: Jul. 4, 2001
(86) PCT No.: PCT/SE01/01544

(30) Foreign Application Priority Data
Jul. 7, 2000 (SE) 0002603-9

(51) Int. Cl. C07D 471/02; A61K 31/4745

(52) U.S. Cl. 514/300; 546/113

(57) ABSTRACT

There is provided compounds of formula (I), wherein R¹, R², R^{3a}, R^{3b} and R⁴¹ to R⁴⁶ have meanings given in the description, which are useful in the prophylaxis and in the treatment of arrhythmias, in particular atrial and ventricular arrhythmias.

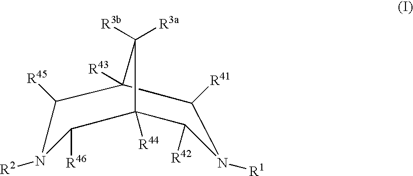

### FIELD OF THE INVENTION

This invention relates to novel pharmaceutically useful compounds, in particular compounds which are useful in the treatment of cardiac arrhythmias.

### BACKGROUND AND PRIOR ART

Cardiac arrhythmias may be defined as abnormalities in the rate, regularity, or site of origin of the cardiac impulse or as disturbances in conduction which causes an abnormal sequence of. activation. Arrhythmias may be classified clinically by means of the presumed site of origin (i.e. as supraventricular, including atrial and atrioventricular, arrhythmias and ventricular arrhythmias) and/or by means of rate (i.e. bradyarrhythmias (slow) and tachyarrhythmias (fast)).

In the treatment of cardiac arrhythmias, the negative outcome in clinical trials (see, for example, the outcome of the Cardiac Arrhythmia Suppression Trial (CAST) reported in New England Journal of Medicine, 321, 406 (1989)) with “traditional” antiarrhythmic drugs, which act primarily by slowing the conduction velocity (class I antiarrhythmic drugs), has prompted drug development towards compounds which selectively delay cardiac repolarization, thus prolonging the QT interval. Class III antiarrhythmic drugs may be defined as drugs which prolong the trans-membrane action potential duration (which can be caused by a block of outward K⁺currents or from an increase of inward ion currents) and refractoriness, without affecting cardiac conduction.

One of the key disadvantages of hitherto known drugs which act by delaying repolarization (class III or otherwise) is that they all are known to exhibit a unique form of proarrhythmia known as torsades de pointes (turning of points), which may, on occasion be fatal. From the point of view of safety, the minimisation of this phenomenon (which has also been .shown to be exhibited as a result of administration of non-cardiac drugs such as phenothiazines, tricyclic antidepressants, antihistamines and antibiotics) is a key problem to be solved in the provision of effective antiarrhythmic drugs.

Antiarrhythmic drugs based on bispidines (3,7-diazabicyclo[3.3.1]nonanes), are known from inter alia international patent applications WO 91/07405 and WO 99/31100, European patent applications 000 074, 301 245, 306 871, 308 843, 461 574 and 665 228, German patent applications DE 24 28 792, DE 26 58 558 and DE 27 44 248 and U.S. Pat. Nos. 3,962,449, 4,556,662, 4,550,112, 4,459,301, 5,468,858 and 5,786,481, as well as journal articles including inter alia: J. Med. Chem. 39, 2559 (1996); Pharmacol. Res. 24, 149 (1991); Circulation, 90, 2032 (1994); Anal. Sci. 9, 429, (1993); and J. Med Chem. 20, 1668 (1977). Known bispidine-based antiarrhythmic compounds include bisaramil (syn-9-(4-chlorobenzoyloxy)-3-methyl-7-ethyl-3,7-diazabicyclo[3.3.1]nonane), tedisamil (3,7-di-(cyclo-propylmethyl)-9,9-tetramethylene-3,7-diazabicyclo[3.3.1]nonane), SAZ-VII-22 (3-(4-chlorobenzoyl)-7-iso-propyl-3,7-diazabicyclo[3.3.1]nonane), SAZ-VII-23 (3-benzoyl-7-iso-propyl-3,7-diazabicyclo[3.3.1]nonane), GLG-V-13 (3-[4-(1H-imidazol-1-yl)benzoyl]-7-iso-propyl-3,7-diazabicyclo-[3.3.1]nonane), KMC-IV-84 (7-[4′-(1H-imidazolo-1-yl)-benzenesulfonyl]-3-iso-propyl-3,7-diazabicyclo[3.3. 1]nonane dihydroperchlorate and ambasilide (3-(4-aminobenzoyl)-7-benzyl-3,7-diazabicyclo[3.3.1]nonane).

Further bispidine compounds are known from inter alia: Eur. J. Med. Chem. 25, 1 (1990); Bull. Polish Acad. Sci. Chem. 34(5-6), 205 (1986); J. Org. Chem. 60, 8148 (1995); Eur. J. Med. Chem.—Chimica Therapeutica 12(4), 301 (1977); Phosphorous, Sulfur and Silicon 123, 385 (1997); J. Org. Chem. 42(6), 937 (1977); and J. Molecular Structure 127, 185 (1985).

We have surprisingly found that a novel group of 3,7-diazabicyclo[3.3.1]-nonane-based compounds exhibit electrophysiological activity, preferably class III electrophysiological activity, and are therefore expected to be usefuil in the treatment of cardiac arrhythmias.

### DISCLOSURE OF THE INVENTION

According to the invention there is provided compounds of formula I,

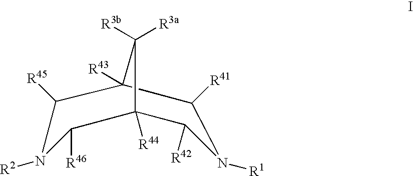

wherein

R¹ represents a structural fragment of formula Ia,

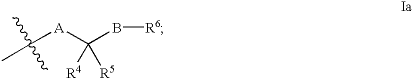

R⁴ represents H, halo, C_{1—4} alkyl, —D—OR⁷, —D—N(R8)R⁹, or R⁴, together with

R⁵, represents ═O;

R⁵ represents H, C₁₋₄ alkyl, or R⁵, together with R⁴, represents ═O;

D represents a direct bond or C₁₋₄ alkylene;

R⁷ represents H, C₁₋₆ alkyl, —E-aryl, —E-Het¹, —C(O)R^{10a}, —C(O)OR^{10b} or —C(O)N(R^{11a})R^{11b};

R⁸ represents H, C₁₋₆ alkyl, —E-aryl, —E-Het¹, —C(O)R^{10a}, —C(O)OR^{10b}, —S(O)₂R^{10c}, —[C(O)]ₙN(R^{11a})R^{11b} or —C(NH)NH₂;

R⁹ represents H, C₁₋₆ alkyl, —E-aryl, or —C(O)R^{10d};

E represents, at each occurrence when used herein, a direct bond or C₁₋₄ alkylene;

R^{10a} to R^{10d} independently represent, at each occurrence when used herein, C₁₋₆ alkyl (optionally substituted and/or terminated by one or more substituents selected from halo, aryl and Het²), aryl, Het³, or R^{10a} and R^{10d} independently represent H;

R^{11a} and R^{11b} independently represent, at each occurrence when used herein, H, C₁₋₆ alkyl (optionally substituted and/or terminated by one or more substituents selected from halo, aryl and Het⁴), aryl, Het⁵, or R^{11a} and R^{11b} together represent C₃₋₇ alkylene, which alkylene group is optionally interrupted by an oxygen atom;

n represents 1 or 2;

A represents —G—, —J—N(R¹²)— or —J—O— (in which latter two groups, J is attached to the bispidine nitrogen atom);

B represents —L—, —L—N(R¹³)—, —N(R¹³)—L—, —L—S(O)ₚ— or —L—O— (in which latter two groups, L is attached to the carbon atom bearing R⁴ and R⁵);

G represents a direct bond or C₁₋₆ alkylene;

J represents C₂₋₆ alkylene;

L represents a direct bond or C₁₋₄ alkylene;

p represents 0, 1 or 2;

R¹² and R¹³ independently represent H or C₁₋₄ alkyl;

R⁶ represents aryl, Het⁶ (both of which groups are optionally substituted and/or terminated (as appropriate) by one or more substituents selected from —OH, cyano, halo, nitro, C₁₋₆ alkyl (optionally terminated by —N(H)C(O)OR^{14a}), C₁₋₆ alkoxy, aryl, Het⁷, —N(R^{15a})R^{15b}, —C(O)R^{15c}, —C(O)OR^{15d}, —C(O)N(R^{15e} )R^{15f}, —N(R^{15g})C(O)R^{15h}, —N(R¹⁵ⁱ)C(O)N(R^{15j})R^{15k}, —N(R^{15m})S(O)₂R^{14b}, —S(O)_{q}R^{14c}, —OS(O)₂R^{14d} and —S(O)₂N(R¹⁵ⁿ)R^{R}^{15p}) when R⁴ and R⁵ together represent ═O, R⁶ may represent C₁₋₆ alkyl; q represents 0, 1 or 2;

R² represents —CN, Het⁸, —C(O)R¹⁶, —C(S)OR¹⁷, —C(S)N(R¹⁸)R¹⁹,

[C(O)]₂N(R^{20a})R^{20b}, —[C(O)]₂OR²¹, —S(O)₂R²², —S(O)₂N(R²³)R²⁴, —C(═N—CN)N(R²⁵)R²⁶, —C(═N—CN)OR²⁷ or C₁₋₁₂ alkyl (which alkyl group is optionally substituted and/or terminated by one or more substituents selected from —C(O)R²⁸, —C(O)N(R^{29a})R^{29b}—N(R³⁰)R³¹, —OR³², —S(O)ᵣR³³, halo, —CN, nitro, aryl and Het⁹);

R¹⁶ represents H, aryl, Het¹⁰ or C₁₋₆ alkyl (which alkyl group is optionally substituted and/or terminated by one or more substituents selected from halo, —OH, —CN, —N(R³⁴)R³⁵, aryl and Het¹¹);

R³⁴ represents, H, C₁₋₆ alkyl, aryl, Het¹², —C(O)R^{36a} or —C(O)OR^{36b};

R¹⁸ represents H, aryl, Het¹³, —C(O)R^{36a}, —C(O)OR^{36b} or C₁₋₆ alkyl (which alkyl group is optionally substituted and/or terminated by one or more substituents selected from halo, —OH, —CN, —C(O)R^{36a} and —C(O)OR^{36b});

R²² represents Het¹⁴, aryl, or C₁₋₆ alkyl (which alkyl group is optionally substituted and/or terminated by one or more substituents selected from halo, —OH, —CN, Het¹⁵ and aryl);

R²³ represents H, C₁₋₆ alkyl, aryl, Het¹⁶, —C(O)R^{36a}, —C(O)OR^{36b} or —C(O)SR^{36b};

R²⁵ represents H or C₁₋₆ alkyl (which alkyl group is optionally substituted and/or terminated by one or more substituents selected from halo, —OH, —CN, C₁₋₆ alkyl (which alkyl group is optionally substituted and/or terminated by one or more substituents selected from C₁₋₄ alkyl and —OH), C₁₋₆ alkoxy and aryl);

R²⁷ represents C₁₋₆ alkyl or aryl;

R²⁸ represents H, C₁₋₆ alkyl, aryl or Het¹⁷;

R^{29a} and R^{29b} independently represent H, C₁₋₆ alkyl, aryl or Het¹⁸;

R³⁰ represents H, C₁₋₆ alkyl, aryl, Het¹⁹, —C(O)R^{37a}, —C(O)OR^{37b} or —C(O)N(R^{37c})R³^{7d};

R³¹ represents H, C₁₋₆ alkyl, aryl or Het²⁰;

R³² represents H, C₁₋₆ alkyl, aryl, Het²¹, —C(O)R^{37a}, —C(O)OR^{37b} or —C(O)N(R^{37c})R^{37d};

R³³ represents C₁₋₆ alkyl, aryl or Het²²;

r represents 0, 1 or 2;

R^{36a} and R^{36b} independently represent, at each occurrence when used herein, C₁₋₆ alkyl, or R^{36a} represents H;

R^{37a} to R^{37d} independently represent, at each occurrence when used herein, C₁₋₆ alkyl, aryl or Het²³, or R^{37a}, R^{37c} and R^{37d} independently represent H;

Het¹ to Het²³ independently represent, at each occurrence when used herein, five- to twelve-membered heterocyclic groups containing one or more heteroatoms selected from oxygen, nitrogen and/or sulfur;

R^{3a} and R^{3b} independently represent H, C₁₋₄ alkyl, —OR^{38a}, —SR^{38b}, —N(R³⁹)R^{38c}, or R^{3a} and R^{3b} together represent C₃₋₅ alkylene, —O—Z—O—, —O—Z—S—or —S—Z—S—;

R³⁹ represents H, C₁₋₆ alkyl or a structural fragment of formula Ia as defined above;

Z represents C₂₋₃ alkylene optionally substituted by one or more C₁₋₄ alkyl groups;

R⁴¹ to R⁴⁶ independently represent H or C₁₋₃ alkyl;

R^{14a} to R^{14d}, R¹⁷ and R²¹ independently represent C₁₆ alkyl;

R^{15a}, R^{15p}, R¹⁹, R^{20a}, R^{20b}, R²⁴, R²⁶, R³⁵ and R^{38a} to R^{38c} independently represent H or C₁₋₆ alkyl;

wherein each aryl and Het (Het¹ to Het²³) group, unless otherwise specified, is optionally substituted;

or a pharmaceutically acceptable derivative thereof;

provided that:

(a) when R¹ represents a structural fragment of formula Ia in which:

R⁴ and R⁵ together represent ═O;

A represents a direct bond;

then B does not represent a direct bond, —N(R¹³)—L— (in which group —N(R¹³)— is attached to the carbon atom bearing R⁴ and R⁵), —N(R¹³)—, —S(O)ₚ— or —O—;

(b) when R⁵ represents H or C₁₋₄ alkyl; and

A represents —J—N(R¹²)— or —J—O—; then B does not represent —N(R³)—L—, —N(R¹³)—, —S(O)ₚ— or —O—;

(c) when R⁴ represents —D—OR⁷, —D—N(R⁸)R⁹ in which D represents a direct bond, then:

(i) A does not represent —J—N(R¹²)— or —J—O—; and

(ii) B does not represent —N(R¹³)—L—, —N(R¹³)—, —S(O)ₚ— or —O—;

(d) when R^{3a} and R^{3b} and both represent H;

and R¹ represents unsubstituted benzyl;

then R² does not represent unsubstituted benzyl or optionally substituted benzoyl; and

(e) the compound is not:

(i) N¹-phenyl-3-(7-benzyl-3,7-diazabicyclo[3.3.1]non-3-yl)-propanamide;

(ii) 3-benzyl-7-[3-(4-cyanophenoxy)-2-hydroxypropyl]-6,8-dimethyl-3,7-diazabicyclo[3.3.1 ]nonane;

(iii) 3-benzyl-7-[3-(4-cyanophenoxy)-2-hydroxypropyl]-6-methyl-3,7-diazabicyclo[3.3.1 ]nonane;

(iv) N-{2-(7-benzyl-3,7-diazabicyclo[3.3.1 ]non-3-yl)-1-[(4-cyanophenoxy)methyl]ethyl} methanesulfonamide;

(v) 3-benzyl-7-[3-(2-propyl-1,3-dioxolan-2-yl)propyl]-3,7-diaza-bicyclo[3.3.1]nonane; or

(vi) 7-benzyl-3,7-diazabicyclo[3.3.1]nonane-3-ethanol;

which compounds are referred to hereinafter as “the compounds of the invention”.

Unless otherwise specified, alkyl groups and alkoxy groups as defined herein may be straight-chain or, when there is a sufficient number (i.e. a minimum of three) of carbon atoms, be branched-chain and/or cyclic.

Further, when there is a sufficient number of carbon atoms (i.e. a minimum of four), such alkyl and alkoxy groups may also be part cyclic/acyclic. Such alkyl and alkoxy groups may also be saturated or, when there is a sufficient number of carbon atoms (i.e. a minimum of two), be unsaturated and/or interrupted by one- or more oxygen and/or sulfur atoms. Unless otherwise specified, alkyl and alkoxy groups may also be substituted by one or more halo, and especially fluoro, atoms.

Unless otherwise specified, alkylene groups as defined herein may be straight-chain or, when there is a sufficient number of carbon atoms (i.e. a minimum of two), be branched-chain. Such alkylene chains may also be saturated or, when there is a sufficient number of carbon atoms (i.e. a minimum of two), be unsaturated and/or interrupted by one or more oxygen and/or sulfur atoms. Unless otherwise specified, alkylene groups may also be substituted by one or more halo atoms.

The term “aryl”, when used herein, includes C₆₋₁₀ aryl groups such as phenyl, naphthyl and the like. Unless otherwise specified, aryl groups may be substituted by one or more substituents including —OH, -cyano, halo, nitro, C₁₋₆ alkyl (optionally terminated by —N(H)C(O)OR^{14a}), C₁₋₆ alkoxy, Het¹, aryl (which aryl group may not be substituted with any further aryl groups), —N(R^{15a})R^{15b}, —C(O)R^{15c}, —C(O)R^{15d}, —C(O)N(R^{15e})R^{15f}, —N(R¹)C(O)R^{15h}, —N(R¹⁵ⁱ)C(O)N(R^{15j})R^{15k}, —N(R^{15m})S(O)₂R^{14b}, —S(O)_{q}R^{14c}, —OS(O)₂R^{14d} and —S(O)₂N(R¹⁵ⁿ)R^{15p}) (wherein Het^{1, R}^{14a} to R^{14d}, R^{15a} to R^{15p} and q are as hereinbefore defined). When substituted, aryl groups are preferably substituted by between one and three substituents.

The term “halo”, when used herein, includes fluoro, chloro, bromo and iodo.

Het (Het¹ to Het²³) groups that may be mentioned include those containing 1 to 4 heteroatoms (selected from the group oxygen, nitrogen and/or sulfur) and in which the total number of atoms in the ring system are between five and twelve. Het (Het¹ to Het²³) groups may be fully saturated, partly unsaturated, wholly aromatic, partly aromatic and/or bicyclic in character. Heterocyclic groups that may be mentioned include benzodioxanyl, benzodioxepanyl, benzodioxolyl, benzofuranyl, benzo-furazanyl, benzimidazolyl, benzomorpholinyl, benzothiophenyl, chromanyl, cinnolinyl, dioxanyl, furanyl, hydantoinyl, imidazolyl, imidazo[1,2-α]pyridinyl, indolyl, isoquinolinyl, isoxazolyl, maleimido, morpholinyl, 2-oxazolidonyl, oxazolyl, phthalazinyl, piperazinyl, piperidinyl, purinyl, pyranyl, pyrazinyl, pyrazolyl, pyridinyl, pyrimidyl, pyrrolidinonyl, pyrrolidinyl, pyrrolinyl, pyrrolyl, quinazolinyl, quinolinyl, sulfolanyl, 3-sulfolenyl, tetrahydropyranyl, tetrahydrofuranyl, tetrazolyl, thiadiazolyl, thiazolyl, thienyl, thiochromanyl, triazolyl and the like. Values of Het¹ that may be mentioned include pyridyl. Values of Het⁶ that may be mentioned include benzodioxanyl, benzomorpholinyl, furanyl, isoquinolinyl, isoxazolyl, 2-oxazolidonyl, piperazinyl, pyrazolyl, pyrrolidinonyl and 1,2,3-thiadiazolyl. Values of Het⁸ that may be mentioned include pyrimidyl, quinazolinyl, tetrazolyl, thiazolyl and 1,2,4-triazolyl. Values of Het⁹ that may be mentioned include benzomorpholinyl, 2-oxazolidonyl and piperazinyl. Values of Het¹⁰ that may be mentioned include furanyl, isoxazolyl, pyrazolyl, pyrrolidinonyl and 1,2,3-thiadiazolyl. Values of Het¹⁴ that may be mentioned include imidazolyl, sulfolanyl, thienyl and quinolinyl. Values of Het¹⁵ that may be mentioned include morpholinyl. Values of Het¹⁷ that may be mentioned include benzomorpholinyl. Values of Het²¹ that may be mentioned include isoquinolinyl.

Unless otherwise specified, Het (Het¹ to Het²³) groups may be substituted by one or more substituents including ═O, —OH, cyano, halo, nitro, C₁₋₆ alkyl (optionally terminated by —N(H)C(O)OR^{14a}), C₁₋₆ alkoxy, Het¹, aryl, —N(R^{15a})R^{15b}, —C(O)R^{15c}, —C(O)OR^{15d}, —C(O)N(R^{15e})R¹⁵, —N(R^{15g})C(O)R^{15h}, —N(R¹⁵ⁱ)C(O)N(R^{5j})R^{15k}, —N(R^{15m})S(O)₂R^{14b}, —S(O)_{q}R^{14b}, —OS(O)₂R^{14d} and —S(O)₂N(R¹⁵ⁿ)R^{15p}) (wherein Het¹, aryl, R^{14a} to R^{14d}, R^{15a} to R^{15p} and q are as hereinbefore defined). When a Het (Het¹ to Het²³) group is substituted by one or more Het¹ and/or aryl group(s), that (those) said Het¹ and/or aryl substituent(s) may not itself (themselves) be substituted by any aryl and/or Het¹ group(s). Substituents on Het (Het¹ to Het²³) groups may, where appropriate, be located on any atom in the ring system including a heteroatom. The point of attachment of Het (Het¹ to Het²³) groups may be via any atom in the ring system including (where appropriate) a heteroatom. Het (Het¹ to Het²³) groups may also be in the N- or S-oxidised form.

Pharmaceutically acceptable derivatives include salts and solvates. Salts which may be mentioned include acid addition salts. Pharmaceutically acceptable derivatives also include, at the 3,7-diazabicyclo[3.3.1]nonane nitrogen, C₁₋₄ alkyl quaternary ammonium salts and N-oxides, provided that when a N-oxide is present:

(a) no Het (Het¹ to Het²³) group contains an unoxidised S-atom;

(b) p does not represent 0 when B represents —L—S(O)ₚ—;

(c) q does not represent 0 when the group —S(O)_{q}R^{14c} is present as a substituent on aryl, Het (Het¹ to Het²³) or R⁶; and/or

(d) r does not represent 0 when the group —S(O)ᵣR³³ is present as a substituent on an alkyl group that R² represents.

The compounds of the invention may exhibit tautomerism. All tautomeric forms and mixtures thereof are included within the scope of the invention.

The compounds of the invention may also contain one or more asymmetric carbon atoms and may therefore exhibit optical and/or diastereoisomerism.

Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention.

Abbreviations are listed at the end of this specification.

Compounds of the invention that may be mentioned include compounds of formula I, as defined above, with the additional provisos that:

(i) when R² represents C₁₋₆ alkyl (optionally substituted by one or two aryl groups), then:

(I) when R⁴ represents H, C₁₋₄ alkyl, —OR⁷, or R⁴, together with R⁵ represents ═O;

R⁷ represents H, C₁₋₆ alkyl or —C(O)R^{10a}; and

R⁶ represents aryl;

then B does not represent —L—; and/or

(II) when A represents a single bond; and

R⁴ and R⁵ together represent ═O;

then R^{3a} and R^{3b} do not both represent C₁₋₄ alkyl or they do not together represent C₃₋₅ alkylene;

(ii) when R² represents —C(O)¹⁶ and the group —A—C(R⁴)(R⁵)—B—represents C₁₋₆ alkylene, then:

(I) R¹⁶ does not represent aryl; and/or

(II) R^{3a} and R^{3a} do not both represent C₁₋₄ alkyl or they do not together represent C₃₋₅ alkylene; and

(iii) when R² represents —S(O)₂R²²; R^{3a} and R^{3b} independently represent H or C₁₋₄ alkyl; and R⁶ represents aryl; then A and B do not simultaneously represent direct bonds, in which above provisos aryl groups, unless otherwise specified, are optionally substituted as described hereinbefore.

Compounds of the invention that may also be mentioned include compounds of formula I, as defined above, with the additional provisos that: when R^{3a} and R^{3b} independently represent H, C₁₋₄ alkyl, OH or N(R³⁹)R^{38c}; and

R³⁹ represents H or C₁₋₆ alkyl, then:

(a) when R² represents CN or C₁₋₆ alkyl optionally substituted by OH, N(R³⁰)R³¹ or Het⁹; R³⁰ and R³¹ independently represent H, C₁₋₆ alkyl or C₃₋₈ cycloalkyl; Het⁹ represents an unsubstituted, saturated 3- to 8-membered heterocycle containing one nitrogen atom (via which atom the heterocyclic group is attached to the rest of the molecule); R⁴ and R⁵ both represent H; and R⁶ represents phenyl substituted in the meta- or para-position (relative to the group B) by CO₂H or NH₂, then:

(i) when A represents a direct bond, C₁₋₆ n-alkylene or —J—O—; and J represents C₂₋₃ n-alkylene; then B does not represent —L—N(R¹³)— or —L—O— (in which latter two groups, L represents a direct bond or C₁₋₄ n-alkylene and is attached to the carbon atom bearing R⁴ and R⁵); and

(ii) when A represents —J—O—; and J represents C₂₋₃ n-alkylene; then B does not represent a direct bond; and

(b) when R⁶ represents Het⁶; Het⁶ represents an unsubstituted, saturated 3- to 8-membered heterocycle containing one nitrogen atom (via which atom the heterocyclic group is attached to the rest of the molecule); and the group —A—C(R⁴)(R⁵)—B— represents C₁₋₆ n-alkylene; then R² does not represent:

(i) C₁₋₆ n-alkyl, which alkyl group is optionally interrupted by O and is terminated by N(R³⁰)R³¹ or R³²; wherein

one of R³⁰ and R³¹ represents H or phenyl substituted in the meta- or para-position (relative to the point of attachment) by CO₂H or NH₂ and the other represents H or C₁₋₆ alkyl; and

R¹² represents H or phenyl substituted in the meta- or para-position (relative to the point of attachment) by CO₂H or NH₂;

(ii) —C(O)R¹⁶, wherein R¹⁶ represents H or phenyl substituted in the meta- or para-position (relative to the point of attachment) by CO₂H or NH₂;

(iii) —S(O)₂R²², wherein R²² represents phenyl substituted in the meta-or-para-position (relative to the point of attachment) by CO₂H or NH₂;

(iv) —S(O)₂N(R²³)R²⁴; wherein

R²³ represents phenyl substituted in the meta- or para-position (relative to the point of attachment) by CO₂H or NH₂; and

R²⁴ represents H or C₁₋₆ alkyl; and

(V) C₃₋₄ n-alkyl, which alkyl group is terminated by phenyl, which phenyl group is substituted in the meta- orpara-position (relative to the point of attachrnent) by CO₂H or NH₂, and which alkyl group is interrupted at the β-position (relative to the point of attachment of the phenyl group) by O.

Further compounds of the invention that may be mentioned include compounds of formula I, as defined above, with the additional proviso that: R⁶ does not represent:

(i) an unsubstituted, saturated 3- to 8-membered heterocycle containing one nitrogen atom (via which atom the heterocyclic group is attached to the rest of the molecule); or

(ii) phenyl substituted in the meta- or para-position (relative to the group B) by CO₂H or NH₂.

Further compounds of the invention that may also be mentioned include those in which R⁶ represents aryl, which group is optionally substituted and/or terminated (as appropriate) by one or more substituents selected from —OH, cyano, halo, nitro, C₁₋₆ alkyl (optionally terminated by —N(H)C(O)OR^{14a}), C₁₋₆ alkoxy, aryl, Het⁷, —C(O)R^{15c}, —C(O)N(R^{15e}) R^{15f}, —N(R^{15g})C(O)R^{15h}, —N(R¹⁵ⁱ)C(O)N(R^{15j})R^{15k}, —N(R¹⁵ⁿ)S(O)₂R^{14b}, —S(O)_{q}R^{14c}, —OS(O)₂R^{14d} and —S(O)₂N(R¹⁵ⁿ)R^{15p}, or, when R⁴ and R⁵ together represent ═O, R⁶ may represent C₁₋₆ alkyl.

Preferred compounds of the invention also include those in which:

R⁴ represents H, C₁₋₂ alkyl, —OR⁷ or N(H)R⁸, or R⁴, together with R⁵, represents ═O;

R⁵ represents H, or R⁵, together with R⁴, represents ═O;

R⁷ represents H, C₁₋₄ alkyl, optionally substituted phenyl, —C(O)R^{10a}, or —C(O)N(R^{11a})R^{11b};

R⁸ represents H, C₁₋₄ alkyl, —C(O)R^{10a}, —C(O)OR¹⁰ or —C(O)N(R^{11a})R^{11b};

R^{10a} and R^{10b} independently represent, at each occurrence when used herein, C₁₋₅ alkyl (optionally substituted and/or terminated by one or more substituents selected from halo and phenyl), optionally substituted phenyl, or R^{10a} represents H;

R^{11a} and R^{11b} independently represent, at each occurrence when used herein, H or C₁₋₅ alkyl (optionally substituted and/or terminated by one or more substituents selected from halo and phenyl);

A represents —G— or —J—N(R¹²)—;

B represents a direct bond, C₁₋₄ alkylene, —L—N(H)—, —L—S(O)₂— or —L—O— (in which latter three groups, L is attached to the carbon atom bearing R⁴ and R⁵);

G represents a direct bond or C₁₋₄ alkylene;

J represents C₂₋₄ alkylene;

L represents C₁₋₄ alkylene;

R⁶ represents phenyl, Het⁶ (both of which groups are optionally substituted by one or more substituents selected from cyano, halo, nitro, C₁₋₄ alkyl, C₁₋₄ alkoxy, optionally substituted phenyl, —N(H)R^{15b}, —C(O)R^{15c}, —C(O)N(H)R^{15f}, N(H)C(O)R^{15h}, —N(H)C(O)N(H)R^{15k}, —N(H)S(O)₂R^{14b}, —S(O)₂R^{14c} and —S(O)₂N(R¹⁵ⁿ)R^{15p}), or, when R⁴ and R⁵ together represent ═O, R⁶ may represent C₁₋₅ alkyl;

R² represents —CN, Het⁸, —C(O)R¹⁶, —C(S)OR¹⁷, —C(S)N(H)R¹⁸, —[C(O)]₂N(H)R^{20b}, —[C(O)]₂OR²¹, —S(O)₂R²², —S(O)₂N(R²³)R²⁴, —C(═N—CN)N(R²⁵)R²⁶, —C(═N—CN)OR²⁷ or C₁₋₆ alkyl (which alkyl group is optionally substituted and/or terminated by one or more substituents selected from —C(O)R²⁸, —C(O)N(H)R^{29b}, —N(R³⁰)R³¹, —OR³², —S(O)₂R³³, halo, —CN, optionally substituted phenyl and Het⁹);

R¹⁶ represents optionally substituted phenyl, Het¹⁰ or C₁₋₆ alkyl (which alkyl group is optionally unsaturated and/or optionally substituted and/or terminated by one or more substituents selected from halo, —CN, —N(H)R³⁴ and optionally substituted phenyl);

R³⁴ represents, H, C₁₋₄ alkyl, —C(O)R^{36a} or —C(O)OR^{36b};

R¹⁸ represents H, —C(O)OR^{36b} or C₁₋₆ alkyl (which alkyl group is optionally substituted and/or terminated by one or more substituents selected from halo and —C(O)OR^{36b});

R²² represents Het¹⁴, optionally substituted phenyl or C₁₋₄ alkyl (which alkyl group is optionally substituted and/or terminated by one or more

substituents selected from halo, Het¹⁵ and optionally substituted phenyl);

R²³ represents H, C₁₋₄ alkyl, —C(O)OR^{36b} or —C(O)SR^{36b};

R²⁵ represents H or C₁₋₆ alkyl (which alkyl group is optionally substituted and/or terminated by one or more substituents selected from halo, —OH, C₁₋₆ alkyl (which alkyl group is optionally substituted and/or terminated by one or more substituents selected from C₁₋₄ alkyl and —OH), C₁₋₄ alkoxy,

naphthyl and optionally substituted phenyl);

R²⁷ represents optionally substituted phenyl;

R²⁸ represents C₁₋₅ alkyl, optionally substituted phenyl or Het¹⁷;

R²⁹ represents H, C₁₋₄ alkyl or optionally substituted phenyl;

R³⁰ represents H, optionally substituted phenyl, —C(O)R^{37a} or —C(O)OR^{37b};

R³¹ represents H, C₁₋₂ alkyl or optionally substituted phenyl;

R³² represents H, C₁₋₄ alkyl (which alkyl group is optionally interrupted by oxygen), optionally substituted phenyl or Het²;

R³³ represents C₁₋₆ alkyl or optionally substituted phenyl;

R^{37a} and R^{37b} independently represent, at each occurrence when used herein, C₁₋₅ alkyl, optionally substituted phenyl, or R^{37a}represents H;

R^{3a} and R^{3b} independently represent H, C₁₋₂ alkyl, —S³⁸, —N(R³⁹)R^{38c}, or

R^{3a} and R^{3b} together represent C₃₋₄ alkylene or —O—Z—O—;

R³⁹ represents H, C₁₋₂ alkyl or a structural fragment of formula Ia;

Z represents C₂₋₃ alkylene;

R⁴¹ to R⁴⁶ independently represent H or C₁₋₂ alkyl;

R^{14b}, R^{14c}, R¹⁷ and R²¹independently represent C₁₄ alkyl;

R^{15b} to R ^{15p}, R^{20b},R²⁴ R²⁶ R^{38b} and R^{38c} independently represent H or C₁₋₅ alkyl;

optional substituents on phenyl groups are one or more substituents selected from cyano, halo, nitro, C₁₋₂ alkyl, C₁₋₂ alkoxy, Het¹, —NH₂, —C(O)R^{15c}, —C(O)N(H)R^{15f}, —N(H)C(O)R^{15h}, —N(H)C(O)N(H)R^{15k}N(H)S(O)²R^{14b} and —S(O)₂N(R¹⁵ⁿ)R^{15p}.

When R^{3a} and/or R^{3b} represent(s) —N(R³⁹)R^{38c} in which R³⁹ represents a structural fragment of formula Ia, preferred compounds of formula I include those in which, in that R³⁹ group:

R⁴ represents H, —OR⁷ or N(H)R⁸, or R⁴, together with R⁵, represents ═O;

R⁵ represents H, or R⁵, together with R⁴, represents ═O;

R⁷ represents H, phenyl (which group is optionally substituted by one to

three methoxy groups), —C(O)CH₃, or —C(O)N(H)—C₁₋₄ alkyl;

R⁸ represents H, —C(O)O—C₁₋₄ alkyl or —C(O)N(H)CH₃;

A represents C₁₋₃ alkylene or —C₂₋₃ alkylene—N(H)—;

B represents a direct bond, —CH₂—, —CH₂—N(H)—, —CH₂—, —S(O)₂—, —CH₂—O— (in which latter three groups, CH₂ is attached to the carbon atom bearing R⁴ and R⁵) or —O—;

R⁶ represents phenyl optionally substituted by up to three substituents (in the para- and/or ortho-positions) selected from cyano, —N(H)C(O)N(H)CH₃, —N(H)S(O)₂CH₃ and —S(O)₂N(CH₃)₂.

More preferred compounds of the invention include those in which:

R⁴ represents H, —OR⁷ or N(H)R⁸, or R⁴, together with R⁵, represents ═O;

R⁵ represents H, or R⁵, together with R⁴, represents ═O;

R⁷ represents H, C₁₋₂ alkyl, optionally substituted phenyl, —C(O)R^{10a}, or —C(O)N(R^{11a})R^{11b};

R⁸ represents H, C₁₋₂ alkyl, —C(O)OR^{10b} or —C(O)N(R¹¹)R^{11b};

R^{10a} and R^{10b} independently represent, at each occurrence when used herein, C₁₋₅ alkyl (optionally substituted or terminated by phenyl), optionally substituted phenyl, or R^{10a} represents H;

R^{11a} and R^{11b} independently represent, at each occurrence when used herein, H or C₁₋₅ alkyl (optionally substituted or terminated by phenyl);

A represents —G—or —J—N(R¹²)—;

B represents a direct bond, C₁₋₄ alkylene, —L—N(H)—, —L—S(O)₂— or —L— (in which latter three groups, L is attached to the carbon atom bearing R⁴ and R⁵);

G represents a direct bond or C₁₋₄ alkylene;

J represents C₂₋₄ alkylene;

L represents C₁₋₄ alkylene;

R⁶ represents phenyl or Het⁶ (which two groups are optionally substituted by one or more substituents selected from cyano, halo, C₁₋₂ alkyl, C₁₋₂ alkoxy, —C(O)R^{15c}, —N(H)C(O)R^{15h}, —N(H)C(O)N(H)R^{15k}, —N(H)S(O)₂R^{14b}, —S(O)₂R^{14c} and —S(O)₂N(R¹⁵ⁿ)R^{15p}).

When R² represents —S(O)₂R²², more preferred compounds of the invention also include those in which:

R⁴, together with R⁵, represents ═O;

A represents C₁₋₄ alkylene;

B represents a direct bond or C₁₋₄ alkylene;

R⁶ represents C₁₋₅ alkyl.

Further preferred compounds of the invention include those in which:

A represents —G—, —J—N(R¹²)— or —J—O— (in which latter two groups, J is attached to the bispidine nitrogen atom);

G represents C₁₋₆ alkylene;

R⁴ represents —D—OR⁷, —D—N(R⁸)R⁹, or R⁴, together with R⁵, represents ═O;

R² represents —CN, Het⁸, —C(O)R¹⁶, —C(S)OR¹⁷, —C(S)N(R¹⁸)R¹⁹, —[C(O)]₂N(R^{20a})R^{20b}, —[C(O)]₂OR²¹, —S(O)₂R²², —S(O)₂N(R²³)R²⁴, —C(═N—CN)N(R²⁵)R²⁶, —C(═N—CN)OR²⁷ or C₁₋₁₂ alkyl (which alkyl group is substituted and/or terminated by one or more substituents selected from —C(O)R²⁸, —C(O)N(R^{29a})R^{29b}, —N(R³⁰)R³¹, —OR³², —S(O)ᵣR³³, halo, —CN, nitro and Het⁹);

R¹⁶ represents H, Het¹⁰ or C₁₋₆ alkyl (which alkyl group is optionally substituted and/or terminated by one or more substituents selected from halo, —OH, —CN, —N(R³⁴)R³⁵, aryl and Het¹¹);

R⁶ represents aryl, Het⁶ (both of which groups are substituted and/or terminated (as appropriate) by one or more substituents selected from —OH, cyano, halo, nitro, C₁₋₆ alkyl (optionally terminated by —N(H)C(O)OR^{14a}), C₁₋₆ alkoxy, aryl, Het⁷, —N(R^{15a})R^{15b}, —C(O)R^{15c}, —C(O)OR^{15d}, —C(O)N(R^{15e})R^{15f}, —N(R^{15g})C(O)R^{15h}, —N(R¹⁵ⁱ)C(O)N(R¹⁵ⁱ)R^{15k}, —N(R^{15m})S(O)₂R^{14b}, —S(O)_{q}R^{14c}, —OS(O)₂R^{14d} and —S(O)₂N(R¹⁵ⁿ))R^{15p}) or, when R⁴ and R⁵ together represent ═O, R⁶ may represent C₁₋₆ alkyl.

Preferred compounds of the invention include the compounds of the examples disclosed hereinafter.

Preparation

According to the invention there is also provided a process for the preparation of compounds of formula I which comprises:

(a) reaction of a corresponding compound of formula II,

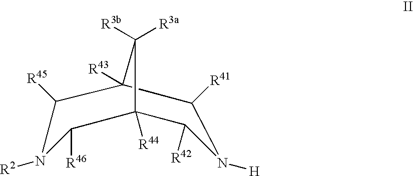

wherein R², R^{3a}, R^{3b} and R⁴¹ to R⁴⁶ are as hereinbefore defined, with a compound of formula III,

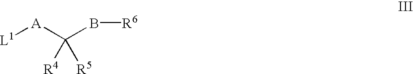

wherein L¹ represents a leaving group (e.g. mesylate, tosylate or halo) and R⁴, R⁵, R⁶, A and B are as hereinbefore defined, for example at between −10° C. and reflux temperature in the presence of a suitable base (e.g. triethylamine or K₂CO₃) and an appropriate organic solvent (e.g. dichloromethane, acetonitrile or DMSO);

(b) for compounds of formula I in which R¹ represents a structural fragment of formula Ia in which A represents C₂ alkylene and R⁴ and R⁴ together represent ═O, reaction of a corresponding compound of formula II, as hereinbefore defined, with a compound of formula IV,

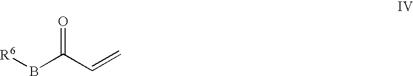

wherein R⁶ and B are as hereinbefore defined, for example at room temperature in the presence of a suitable organic solvent (e.g. ethanol);

(c) for compounds of formula I in which R^{3a} or R^{3b} represents —N(R³⁹)R^{38c} and R³⁹ represents a structural fragment of formula Ia, reaction of a corresponding compound of formula I in which R^{3a} or R^{3b} (as appropriate) represents —N(H)R^{38c}, wherein R^{38c} is as hereinbefore defined, with a compound of formula III as hereinbefore defined, for example under conditions described hereinbefore (see process step (a));

(d) for compounds of formula I in which R¹ represents a fragment of formula Ia in which A represents CH₂ and R⁴ represents —OH or —N(H)R⁸, reaction of a corresponding compound of formula II, as hereinbefore defined, with a compound of formula V,

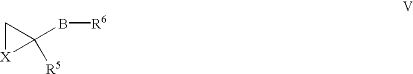

wherein X represents O or N(R⁸) and R⁵, R⁶, R⁸ and B are as hereinbefore defined, for example at elevated temperature (e.g. 60° C. to reflux) in the presence of a suitable solvent (e.g. a lower alkyl alcohol (e.g. IPA), acetonitrile, or a mixture of a lower alkyl alcohol and water);

(e) for compounds of formula I in which R^{3a} or R^{3b} represents —N(R³⁹)R^{38c} and R³⁹ represents a structural fragment of formula Ia in which A represents CH₂ and R⁴ represents —OH or —N(H)R⁸, reaction of a corresponding compound of formula I in which R^{3a} or R^{3b} (as appropriate) represents —N(H)R^{38c}, wherein R^{38c} is as hereinbefore defined, with a compound of formula V as hereinbefore defined, for example under conditions described hereinbefore (see process step (d));

(f) for compounds of formula I in which A represents C₁₋₆ alkylene, B represents C₁₋₄ alkylene and R⁴ and R⁵ both represent H, reduction of a corresponding compound of formula I in which R⁴ and R⁵ together represent ═O, in the presence of a suitable reducing agent and under appropriate reaction conditions, for example by activating the relevant C═O group using an appropriate agent (such as tosylhydrazine) in the presence of a suitable reducing agent (e.g. sodium borohydride or sodium cyanoborohydride) and an appropriate organic solvent (e.g. a lower (e.g. C₁₋₆) alkyl alcohol);

(g) for compounds of formula I in which R⁴ and R⁵ both represent H and (1) A represents a single bond or —J—N(R¹²) and B represents C₁₋₄ alkylene, or (2) A represents C₁₋₆ alkylene and B represents N(R¹³) or —N(R¹³)—L—, reduction of a corresponding compound of formula I in which R⁴ and R⁵ together represent ═O, in the presence of a suitable reducing agent (e.g. LiAlH₄) and an appropriate solvent (e.g. THF);

(h) for compounds of formula I in which A represents C₁₋₆ alkylene, B represents a direct bond, C₁₋₄ alkylene, —L—N(R¹³)—, —L—S(O)ₚ— or —L—O— (in which latter three groups L represents C₁₋₄ alkylene), R⁴ represents OH and R⁵ represents H, reduction of a corresponding compound of formula I in which R⁴ and R⁵ together represent ═O, in the presence of a suitable reducing agent (e.g. NaBH₄) and an appropriate organic solvent (e.g. THF);

(i) for compounds of formula I in which R^{3a} and R^{3b} both represent H, reduction of a corresponding compound of formula VI,

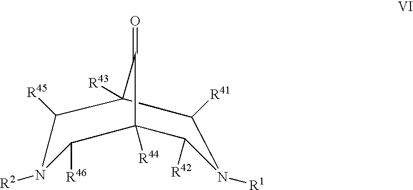

wherein R¹, R² and R⁴¹ to R⁴⁶ are as hereinbefore defined, and in which the bridgehead C═O group may be activated using an appropriate agent, such as tosylhydrazine, in the presence of a suitable reducing agent (e.g. sodium borohydride, sodium cyanoborohydride) and an appropriate organic solvent (e.g. a lower alkyl alcohol), or under standard Wolff-Kisclmer conditions known to those skilled in the art; when the C═O group is activated, the activation step may be carried out at between room and reflux temperature in the presence of an appropriate organic solvent (e.g. a lower alkyl alcohol such as methanol, ethanol or IPA), whereafter the reducing agent may be added to the reaction mixture and the reduction carried out at between 60° C. and reflux, advantageously in the presence of a suitable organic acid (e.g. acetic acid);

(j) for compounds of formula I in which one of R^{3a} and R^{3b} represents H, and the other represents —OH, reduction of a corresponding compound of formula VI, as hereinbefore defined, in the presence of a mild reducing agent, e.g. sodium borohydride, and an appropriate organic solvent (e.g. a lower alcohol such as methanol or ethanol);

(k) for compounds of formula I in which R^{3a} and R^{3b} both represent —OR^{38a} or —SR^{38b}, or in which R^{3a} and R^{3b} together represent —O—Z—O—, —O—Z—S— or —S—Z—S—, reaction of a corresponding compound of formula VI, as hereinbefore defined, with a compound of formula HOR^{38a}, HSR^{38b}, HO—Z—OH, HO—Z—SH or HS—Z—SH (as appropriate), wherein R^{38a}, R^{38b} and Z are as hereinbefore defined, under appropriate reaction conditions, for example by refluxing in the presence of a suitable protic or Lewis acid catalyst (e.g. pTSA, trimethylsilyl chloride or boron trifluoride) and an appropriate organic solvent (e.g. toluene or diethyl ether);

(1) for compounds of formula I in which one of R^{3a} and R^{3b} represents —NH₂ and the other represents H, reduction of a compound of formula VII,

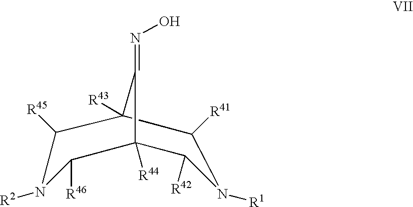

wherein R¹, R² and R⁴¹ to R⁴⁶ are as hereinbefore defined, in the presence of a suitable reducing agent (e.g. LiAlH₄), for example under conditions that are well known to those skilled in the art;

(m) for compounds of formula I in which one or both of R^{3a} and R^{3b} represent —N(R³⁹)R^{38c} in which one or both of R³⁹ and R^{38c} represents C₁₋₆ alkyl, alkylation of a corresponding compound of formula I in which R^{3a}and/or R^{3b} represent —N(R³⁹)R^{38c} (as appropriate) in which R³⁹ and/or R^{38c} (as appropriate) represent H, using a compound of formula VIII,

R^{a}—L¹ VIII

wherein R^{a} represents C₁₋₆ alkyl and L¹ is as hereinbefore defined, for example under conditions that are well known to those skilled in the art;

(n) for compounds of formula I in which R¹ represents a structural fragment of formula Ia in which B represents —L—O—, reaction of a compound of formula IX,

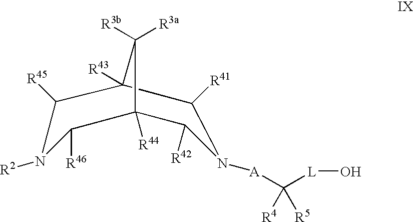

wherein R², R³, R^{3b}, R⁴, R⁵, R⁴¹ to R⁴⁶, A and L are as hereinbefore defined, with a compound of formula X,

R⁶OH X

in which R⁶ is as hereinbefore defined, for example under Mitsunobu-type conditions e.g. at between ambient (e.g. 25° C.) and reflux temperature in the presence of a tertiary phosphine (e.g. tributylphosphine or triphenylphosphine), an azodicarboxylate derivative (e.g. diethylazodicarboxylate or 1,1′-(azodicarbonyl)dipiperidine) and an appropriate organic solvent (e.g. dichloromethane or toluene);

(o) for compounds of formula I in which R¹ represents a structural fragment of formula Ia in which A represents C₁₋₆ alkylene and B represents —N(R¹³)—L— (wherein the group —N(R¹³)— is attached to the carbon atom bearing R⁴ and R⁵), reaction of a compound of formula XI,

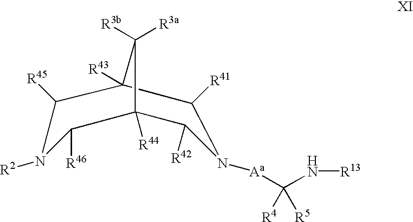

wherein A^{a} represents C₁₋₆ alkylene and R², R^{3a}, R^{3b}, R⁴, R⁵, R¹³ and R⁴¹ to R⁴⁶ are as hereinbefore defined with a compound of formula XII,

R⁶—L—L² XII

wherein L² represents a leaving group such as halo, alkane sulfonate, perfluoroalkane sulfonate or arenesulfonate, and R⁶ and L are as hereinbefore defined, for example at 40° C. in the presence of a suitable organic solvent (e.g. acetonitrile);

(p) for compounds of formula I in which R¹ represents a structural fragment of formula Ia in which R⁴ represents —D—NH₂, reduction of a corresponding compound of formula XIII,

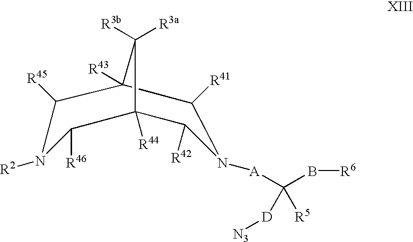

wherein R², R^{3a}, R^{3b}, R⁵, R⁶, R⁴¹ to R⁴⁶, A, B and D are as hereinbefore defined, for example by hydrogenation at a suitable pressure in the presence of a suitable catalyst (e.g. palladium on carbon) and an appropriate solvent (e.g. a water-ethanol mixture);

(q) for compounds of formula I in which R⁴ represents —D—N(R⁹)C(O)NH(R^{11b}), reaction of a corresponding compound of formula I in which R⁴ represents —D—N(R⁹)H with a compound of formula XIV,

R^{11b}N═C═O XIV

wherein R^{11b} is as hereinbefore defined, for example at ambient temperature (e.g. 25° C.) in the presence of a suitable solvent (e.g. benzene);

(r) for compounds of formula I in which R⁴ represents —D—N(H)[C(O)]₂NH₂, reaction of a corresponding compound of formula I in which R⁴ represents —D—NH₂ with oxalic acid diamide, for example at between −10 and 25° C. in the presence of a suitable coupling agent (e.g. 1-(3-dimethylaminopropyl)-3-ethylcarbodiimide), an appropriate activating agent (e.g. 1-hydroxybenzo-triazole), a suitable base (e.g. triethylamine) and a reaction-inert organic solvent (e.g. DMF);

(s) for compounds of formula I in which R⁴ represents —D—N(R⁸)R⁹, wherein R⁸ and R⁹ are as hereinbefore defined, provided that R⁸ does not represent H, reaction of a corresponding compound of formula I, in which R⁴ represents —D—N(H)R⁹ with a compound of formula XV,

R^{8a}—L³ XV

wherein R^{8a} represents R⁸ as hereinbefore defined except that it does not represent H, and L³ represents a leaving group such as halo (e.g. chloro or bromo), p-nitrophenolate, C₁₋₄ alkoxide, C₁₋₄ alkylthiolate, —OC(O)R^{10a}, —OC(O)OR^{10b}, or —OS(O)₂R^{10c}, wherein R^{10a} to R^{10c} are as hereinbefore defined, for example under conditions that are well known to those skilled in the art;

(t) for compounds of formula I in which R⁴ represents —D—OR⁷ in which R⁷ represents C₁₋₆ alkyl, —E-aryl or —E-Het¹, reaction of a corresponding compound of formula I in which R⁴ represents —D—OH with a compound of formula XVI,

R^{7a}OH XVI

wherein R^{7a} represents C₁₋₆ alky, —E-aryl or —E-Het¹, wherein Het¹ is as hereinbefore defined, for example at between ambient (e.g. 25° C.) and reflux temperature, under Mitsunobu-type conditions (i.e. in the presence of e.g. triphenylphosphine, an azodicarboxylate derivative (e.g. 1,1′-(azodicarbonyl)dipiperidine) and a suitable organic solvent (e.g. dichloromethane));

(u) for compounds of formula I in which R¹ represents a structural fragment of formula Ia in which R⁴ represents —D—OR⁷ (in which R⁷ represents C₁₋₆ alkyl, —E-aryl or —E-Het¹), reaction of a corresponding compound of formula XVII,

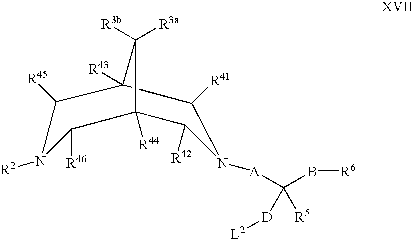

wherein L², R², R^{3a}, R^{3b}, R⁵, R⁶, R⁴¹ to R⁴⁶, A, B and D are as hereinbefore defined with a compound of formula XVI as hereinbefore defined, for example at between ambient (e.g. 25° C.) and reflux temperature, under Williamson-type conditions (i.e. in the presence of an appropriate base (e.g. KOH or NaH) and a suitable organic solvent (e.g. dimethylsulfoxide or DMF));

(v) for compounds of formula I in which R⁴ represents —D—OR⁷, wherein R⁷ is as hereinbefore defined, provided that it does not represent H, reaction of a corresponding compound of formula I in which R⁴ represents —D—OH with a compound of formula XVIII,

R^{7b}—L⁴ XVIII

wherein R^{7b} represents R⁷ as hereinbefore defined, except that it does not represent H, and L⁴ represents a leaving group such as OH, halo, alkane sulfonate, arene sulfonate or —OC(O)R^{10a}, wherein R^{10a} is as hereinbefore defined, for example at between room and reflux temperature, optionally in the presence of a reaction-inert organic solvent (e.g. THF or CH₂Cl₂), a suitable base (e.g. triethylamine or K₂CO₃) and/or an appropriate coupling agent (e.g. 1,3-dicyclohexylcarbodiimide or 1-(3-dimethylaminopropyl)-3-ethylcarbodiimide, optionally combined with a suitable catalyst such as 4-dimethylaminopyridine) (for example, when R^{7b} represents —C(O)R^{10a} and L⁴ represents OH, this reaction may be performed at ambient temperature (e.g. 25° C.) in the presence of a coupling agent such as 1-(3-dimethylamino-propyl)-3-ethylcarbodiimide, an appropriate catalyst such as 4-(dimethyl-amino)pyridine and a solvent such as THF);

(w) for compounds of formula I in which R⁴ represents halo, substitution of a corresponding compound of formula I in which R⁴ represents —OH, using an appropriate halogenating agent (e.g., for compounds in which R⁴ represents fluoro, reaction with diethylaminosulfurtrifluoride);

(x) reaction of a corresponding compound of formula XIX,

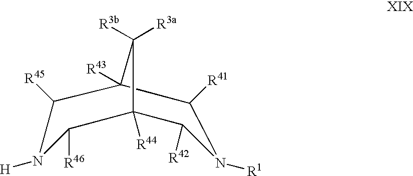

wherein R¹, R^{3a}, R^{3b} and R⁴¹ to R⁴⁶ are as hereinbefore defined, with a compound of formula XX,

R²—L⁵ XX

wherein L⁵ represents a leaving group such as halo, OH, alkane sulfonate, arene sulfonate, C₁₋₄ alkoxy, phenoxy, —OC(O)R¹⁶, —OC(O)OR²¹, or —OS(O)₂R²², and R², R¹⁶, R²¹ and R²² are as hereinbefore defined, for example at between −10° C. and reflux temperature, optionally in the presence of a suitable solvent (e.g. CDCl₃, CH₃CN, 2-propanol, diethyl ether, or mixtures thereof) and/or an appropriate base (e.g. K₂CO₃, pyridine or triethylamine);

(y) for compounds of formula I in which R² represent C₁₋₂ alkyl, which alkyl group is substituted at the C-2 carbon (relative to the bispidine nitrogen) with OH or N(H)R³⁰, and is otherwise optionally substituted with one or more further substituents as specified hereinbefore for R², reaction of a compound of formula XIX as hereinbefore defined with a compound of formula XXA

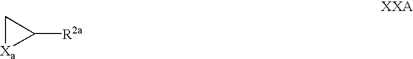

wherein Xa represents O or N(R³⁰) and R^{2a} represents C₁₋₄ alkyl, optionally substituted with one or more substituents as specified hereinbefore for R², for example as described hereinbefore for preparation of compounds of formula I (process step (d));

(z) for compounds of formula I in which R² represents tetrazol-5-yl, reaction of a corresponding compound of formula I in which R² represents —CN with a suitable source of the azide ion (e.g. sodium azide), for example at elevated temperature (e.g. 100° C.) in the presence of an appropriate solvent (e.g. DMF) and optionally in the presence of a suitable proton source (e.g. NH₄Cl);

(aa) for compounds of formula I which are bispidine-nitrogen N-oxide derivatives, oxidation of the corresponding bispidine nitrogen of a corresponding compound of formula I, in the presence of a suitable oxidising agent (e.g. mCPBA), for example at 0°C. in the presence of a suitable organic solvent (e.g. DCM);

(ab) for compounds of formula I which are C₁₋₄ alkyl quaternary ammonium salt derivatives, in which the alkyl group is attached to a bispidine nitrogen, reaction, at the bispidine nitrogen, of a corresponding compound of formula I with a compound of formula XXI,

R^{b}—L² XXI

wherein R^{b} represents C₁₋₄ alkyl and L² is as hereinbefore defined, for example at room temperature in the presence of an appropriate organic solvent (e.g. DMF), followed by purification (using e.g. HPLC) in the presence of a suitable counter-ion provider (e.g. NH₄OAc);

(ac) conversion of one substituent on R⁶ to another using techniques well known to those skilled in the art; or

(ad) conversion of one R² group to another using techniques well known to those skilled in the art.

Compounds of formula II may be prepared by reaction of a corresponding compound of formula XXII,

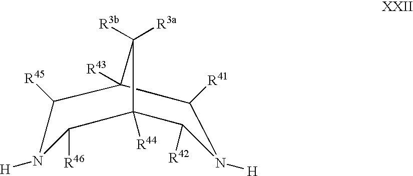

wherein R^{3a}, R^{3b} and R⁴¹ to R⁴⁶ are as hereinbefore defined, with a compound of formula XX as hereinbefore defined, for example as described hereinbefore for synthesis of compounds of formula I (process step (x)).

Compounds of formula II in which R^{3a} and R^{3b} both represent H may be prepared by reduction of a corresponding compound of formula XXIII,

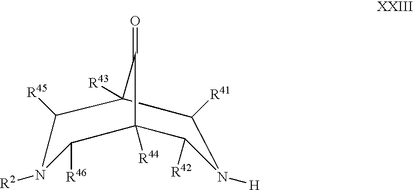

wherein R² and R⁴¹ to R⁴⁶ are as hereinbefore defined, and in which the C═O group may be activated using an appropriate agent, such as tosylhydrazine, for example as described hereinbefore for the synthesis of compounds of formula I (process step (i)).

Compounds of formula II in which one of R^{3a} and R^{3b} represents —OH and the other represents C₁₋₄ alkyl may be prepared by reaction of a compound of formula XXIII, or a protected derivative thereof, with a compound of formula XXIV,

R^{alk}—Mg-Hal XXIV

wherein R^{alk} represents C₁₋₄ alkyl and Hal represents chloro, bromo or iodo, for example at between −25° C. and ambient temperature in the presence of a suitable solvent (e.g. diethyl ether).

Compounds of formula III may be prepared by standard techniques. For example compounds of formula III in which:

(1) B represents —L—O— may be prepared by coupling a Compound of formula X, as hereinbefore defined, to a compound of formula XXV,

L⁶—L—C(R⁴)(R⁵)—A—L¹ XXV

wherein L⁶ represents a suitable leaving group (e.g. halo) and R⁴, R⁵, A, L and L¹ are as hereinbefore defined; or

(2) B represents —N(R¹³)—L— and R⁴ and R⁵ together represent ═O may be prepared by coupling a compound of formula XXVI,

R⁶—L—N(R¹³)H XXVI

wherein R⁶, R¹³ and L are as hereinbefore defined, to a compound of formula XXVII,

L⁶—C(O)—A—L¹ XXVII

wherein L⁶, A and L¹ are as hereinbefore defined;

in both cases, under conditions which are well known to those skilled in the art.

Compounds of formula III in which A represents C₂-alkylene and R⁴ represents —OR⁷, in which R⁷ represents C₁₋₆ alkyl, —E-aryl or —E-Het¹ may alternatively be prepared by reaction of a compound of formula XVI as hereinbefore defined with a compound of formula XXVIII,

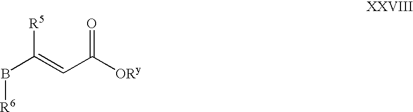

wherein R ^{y} represents C₁₋₄, alkyl or aryl (which two groups are optionally substituted with one or more substituents selected from C₁₋₄ alkyl or halo) and R⁵, R⁶ and B are as hereinbefore defined, for example at between ambient temperature (e.g. 25° C.) and reflux temperature in the presence of a suitable base (e.g. K₂CO₃) and an appropriate organic solvent (e.g. acetonitrile), followed by conversion of the ester functionality to an L¹ group (in which L¹ is as hereinbefore defined), under conditions that are well known to those skilled in the art.

Compounds of formula III in which A represents C₂₋₆ alkylene may be prepared by reduction of a corresponding compound of formula XXIX,

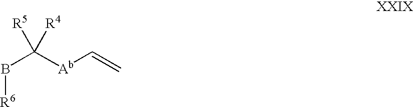

wherein A^{b} represents a direct bond or C₁₋₄ alkylene, and R⁴, R⁵, R⁶ and B are as hereinbefore defined, with a suitable borane or borane-Lewis base complex (e.g. borane-dimethyl sulfide) in the presence of an appropriate solvent (e.g. diethyl ether, THF, or a mixture thereof), followed by oxidation of the resulting borane adduct with a suitable oxidising agent (e.g. sodium perborate) and then conversion of the resulting OH group to an L¹ group under conditions known to those skilled in the art.

Compounds of formula III in which A represents C₂₋₆ alkylene and B represents —L—N(R¹³)— (wherein L represents C₁₋₄ alkylene) may be prepared by coupling a compound of formula XXX,

R⁶—L⁶ XXX

wherein R⁶ and L⁶ are as hereinbefore defined, with a compound of formula XXXI,

HN(R¹³)—L^{a}—C(R⁴)(R⁵)—A—OH XXXI

wherein L^{a} represents C₁₋₄ alkylene, A^{c} represents C₂₋₆ alkylene, and R⁴, R⁵ and R¹³ are as hereinbefore defined, for example at between room and reflux temperature, optionally in the presence of a suitable solvent and/or an appropriate base, followed by conversion of the OH group to an L¹ group under conditions known to those skilled in the art.

Compounds of formula III in which B represents —L—S(O)— or —L—S(O)₂— may be prepared by oxidation of corresponding compounds of formula III in which B represents —L—S—, wherein L is as hereinbefore defined, in the presence of an appropriate amount of a suitable oxidising agent (e.g. mCPBA) and an appropriate organic solvent.

Compounds of formula V may be prepared in accordance with techniques which are known to those skilled in the art. For example, compounds of formula V in which:

(1) B represents —CH₂O— and X represents O may be prepared by reaction of a compound of formula X, as hereinbefore defined, with a compound of formula XXXII,

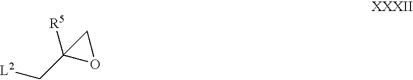

wherein R⁵ and L² are as hereinbefore defined, for example at elevated temperature (e.g. between 60° C. and reflux temperature) in the presence of a suitable base (e.g. K₂CO₃ or NaOH) and an appropriate organic solvent (e.g. acetonitrile or toluene/water), or as otherwise described in the prior art;

(2) R⁵ represents H and X represents O may be prepared by reduction of a compound of formula XXXIII,

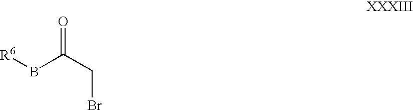

wherein R⁶ and B are as hereinbefore defined, for example at between −15° C. and room temperature in the presence of a suitable reducing agent (e.g. NaBH₄) and an appropriate organic solvent (e.g. THF), followed by an internal displacement reaction in the resultant intermediate, for example at room temperature in the presence of a suitable base (e.g. K₂CO₃) and an appropriate organic solvent (e.g. acetonitrile);

(3) B represents —L—, —L—N(R¹³)—, —L—S(O)₂— or —L—O— (in all four of which groups L represents C₁₋₄ alkylene) and X represents O may be prepared by oxidation of a compound of formula XXXIV,

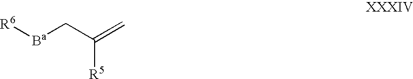

wherein B^{a} represents —L—, —L—N(R¹³)—, —L—S(O)₂— or —L—O— (in all four of which groups L represents a single bond or C₁₋₃ alkylene), and R⁵, R⁶ and R¹³ are as hereinbefore defined, in the presence of a suitable oxidising agent (e.g. mCPBA), for example by refluxing in the presence of a suitable organic solvent (e.g. DCM); or

(4) B represents —L—O— (in which group L represents C₁₋₄ alkylene) and X represents N(R⁸) (wherein R⁸ represents —C(O)OR^{10b} or —S(O)₂R^{10c}) may be prepared by cyclisation of a compound of formula XXXV,

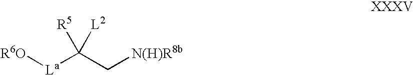

wherein R^{8b} represents —C(O)OR^{10b} or —S(O)₂R^{10c} and R⁵, R⁶, R^{10b}, R^{10c}, L^{a} and L² are as hereinbefore defined, for example at between 0° C. and reflux temperature in the presence of a suitable base (e.g. sodium hydroxide), an appropriate solvent (e.g. dichloromethane, water, or a mixture thereof) and, if necessary a phase transfer catalyst (such as tetrabutylammonium hydrogensulfate).

Compounds of formula VII may be prepared by reaction of a corresponding compound of formula VI with hydroxylamine, for example at elevated temperature (e.g. at reflux) in the presence of a suitable organic solvent (e.g. methanol).

Compounds of formulae IX, XI, XIII and XVII may be prepared in a similar fashion to compounds of formula I (see, for example, process steps (a), (b) and (x)).

Compounds of formula XIII may alternatively be prepared by reaction of a corresponding compound of formula I in which R⁴ represents —D—OH, with a compound of formula XXXVI,

R^{y}S(O)₂Cl XXXVI

wherein R^{y} is as hereinbefore defined, for example at between −10 and 25° C. in the presence of a suitable solvent (e.g. dichloromethane), followed by reaction with a suitable source of the azide ion (e.g. sodium azide) for example at between ambient and reflux temperature in the presence of an appropriate solvent (e.g. DMF) and a suitable base (e.g. NaHCO₃).

Compounds of formula XVII may alternatively be prepared by replacement of the OH group of a compound of formula I in which R⁴ represents —D—OH with an L² group under conditions that are well known to those skilled in the art.

Compounds of formula XIX may be prepared by reaction of a corresponding compound of formula XXII, as hereinbefore defined, with a compound of formula III, as hereinbefore defined.

Compounds of formula XIX in which A represents C₂ alkylene and R⁴ and R⁵ together represent ═O may be prepared by reaction of a corresponding compound of formula XXII, as hereinbefore defined, with a compound of formula IV, as hereinbefore defined, for example as described hereinbefore for synthesis of compounds of formula I (process step (b)).

Compounds of formula XIX in which A represents CH₂ and R⁴ represents —OH or —N(H)R⁸ may be prepared by reaction of a corresponding compound of formula XXII, as hereinbefore defined, with a compound of formula V as hereinbefore defined, for example as described hereinbefore for synthesis of compounds of formula I (process step (d)).

Compounds of formula XIX in which R^{3a} and R^{3b} both represent H may. alternatively be prepared by reduction of a corresponding compound of formula XXXVII,

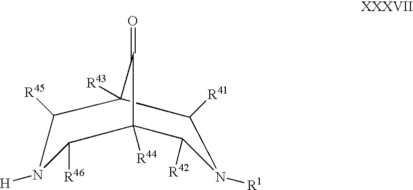

wherein R¹ and R⁴¹ to R⁴⁶ are as hereinbefore defined, and in which the C═O group may be activated using an appropriate agent, such as tosylhydrazine, for example as described hereinbefore for the synthesis of compounds of formula I (process step (i)).

Compounds of formulae II and XIX in which one or more of R⁴¹, R⁴², R⁴⁵ and/or R⁴⁶ represent C₁₋₃ alkyl may alternatively be prepared by reaction of a compound of formula II or XIX (as appropriate) in which R⁴¹, R⁴², R⁴⁵ and/or R⁴⁶ (as appropriate) represent H, with an appropriate alkylating agent (e.g. dimethyl sulfate), for example in the presence of a suitable strong base (e.g. s-BuLi), N,N,N′,N′-tetramethylethylenediamine and a reaction-inert solvent (e.g. THF).

Compounds of formula XX in which R² represents —C(═N—CN)N(R²⁵)R²⁶ and L⁵ represents phenoxy may be prepared by reaction of a corresponding compound of formula XXXVIII,

H—N(R²⁵)R²⁶ XXXVIII,

wherein R²⁵ and R²⁶ are as hereinbefore defined, with diphenyl cyanocarbonimidoate, for example at between −10° C. and room temperature in the presence of an appropriate solvent (e.g. isopropanol).

Compounds of formula XXII are known in the literature or are readily available using known techniques. For example, compounds of formula XXII in which R^{3a} and R^{3b} together represent C₃₋₅ alkylene, —O—Z—O—, —O—Z—S— or —S—Z—S— and R⁴¹ to R⁴⁶ all represent H, may be prepared by reduction of a compound of formula XXXIX,

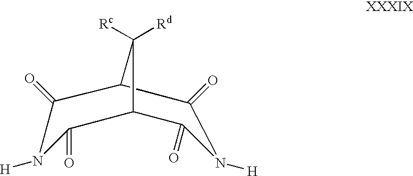

wherein R^{c} and R^{d} together represent C₃₋₅ alkylene, —O—Z—O—, —O—Z—S— or —S—Z—S—, wherein Z is as hereinbefore defined, in the presence of a suitable reducing agent (e.g. LiAlH₄) under conditions that are well known to those skilled in the art.

Compounds of formula XXIX in which B represents C₁₋₄ alkylene may be prepared by coupling a compound of formula XL,

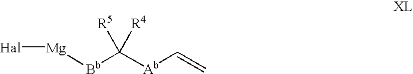

wherein B represents C₁₋₄ alkylene and Hal, A^{b}, R⁴ and R⁵ are as hereinbefore defined, with a compound of formula XXX, as hereinbefore defined, for example at between −25° C. and room temperature in the presence of a suitable zinc(II) salt (e.g. anhydrous ZnBr₂), an appropriate catalyst (e.g. Pd(PPh₃)₄) and a reaction-inert organic solvent (e.g. THF, toluene or diethyl ether).

Compounds of formulae VI, XXIII and XXXVII (in which, in all cases, R⁴¹ and R⁴² both represent H), may be prepared, advantageously, by reaction of (as appropriate) either (i) a compound of formula XLI,

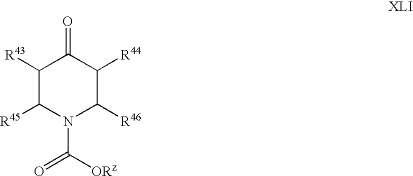

wherein R^{Z} represents C₁₋₁₀ alkyl or C₁₋₃ alkylaryl (e.g. alkylphenyl, such as benzyl) and R⁴³ to R⁴⁶ are as hereinbefore defined, or (ii) 4-piperidone (or a protected derivative thereof), with (as appropriate) either (1) a compound of formula XLII,

R⁶—B—C(R⁴)(R⁵)—A—NH₂ XLII

wherein R⁴, R⁵, R⁶, A and B are as hereinbefore defined, or (2) NH₃ (or a protected (e.g. benzyl) derivative thereof), in all cases in the presence of a formaldehyde (i.e. an appropriate source of formaldehyde, such as paraformaldehyde or formalin solution) and, in the case of compounds of formulae VI and XXIII, conversion of the C(O)OR^{Z} group in the resultant intermediate to an R² group using techniques such as those described herein (e.g. removal of the C(O)OR^{Z} group followed by carrying out a coupling, e.g. according to process step (x) above).

The formation of compounds of formulae VI, XXIII and XXXVII may be carried out in this way for example at between room temperature and reflux (depending upon the concentration of the reactants) in the presence of an appropriate solvent (e.g. ethanol or methanol) and, preferably, in the presence of an organic acid (e.g. a C₁₋₆ carboxylic acid, especially acetic acid).

It will be also appreciated by those skilled in the art that compounds of formula XXII in which R^{3a} and R^{3b} both represent H may also be prepared via this method (i.e. by reaction of a 4-piperidone (or a protected derivative thereof) with NH₃ (or a protected derivative thereof) in the presence of a formaldehyde), provided that the intermediate so formed is subsequently reduced under appropriate reaction conditions.

The skilled person will also appreciate that this process may also be used to prepare compounds of formula I in which R⁴⁵ and R⁴⁶ are H, and R⁴¹ and/or R⁴² are other than H, for example by:

(i) reacting a compound of formula XLI in which R⁴⁵ and/or R⁴⁶ is/are other than H with, for example, benzylamine or a derivative thereof;

(ii) removal of the —C(O)OR^{Z} unit;

(iii) reaction at the free bispidine nitrogen of the resultant compound with a compound of formula III, IV or V (as appropriate), as hereinbefore defined;

(iv) removal of the benzyl protecting group; and

(v) reaction at the free bispidine nitrogen of the resultant compound with a compound of formula XX as hereinbefore defined, under conditions well known to those skilled in the art including those described hereinbefore. This reaction will be accompanied by, at some point, conversion of the bridgehead carbonyl functionality to the desired R^{3a}/R^{3b} groups.

Compounds of formula XXXIX may be prepared in accordance with techniques which are well known to those skilled in the art. For example, compounds of formula XXXIX in which R^{c} and R^{d} together represent C₃₋₅ alkylene may be prepared by reaction of a compound of formula XLIII,

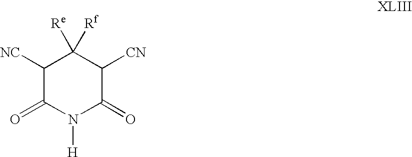

wherein R^{e} and R^{f} together represent C₃₋₅ alkylene, with a mixture of phosphoric acid and sulfuric acid, for example at 120° C.

Compounds of formula XLII are well known in the literature or are readily is available using known techniques. For example, compounds of formula XLII in which R⁴ represents OH, R⁵ represents H and A represents CH₂ may be prepared by reaction of a corresponding compound of formula V wherein R⁵ represents H and X represents O with ammonium hydroxide under conditions which are well known to those skilled in the art.

Compounds of formulae IV, VIII, X, XII, XIV, XV, XVI, XVIII, XX (in which R² represents other than —C(═N—CN)N(R²⁵)R²⁶ and/or L⁵ represents other than phenoxy), XXA, XXI, XXIV, XXV, XXVI, XXVII, XXVIII, XXX, XXXI, XXXII, XXXIII, XXXIV, XXXV, XXXVI, XXXVIII, XL, XLI, XLIII and derivatives thereof, are either commercially available, are known in the literature, or may be obtained either by analogy with the processes described herein, or by conventional synthetic procedures, in accordance with standard techniques, from readily available starting materials using appropriate reagents and reaction conditions.

Substituents on the aryl (e.g. phenyl), and (if appropriate) heterocyclic, group(s) in compounds defined herein may be converted to other claimed substituents using techniques well known to those skilled in the art. For example, hydroxy may be converted to alkoxy, phenyl may be halogenated to give halophenyl, nitro may be reduced to give amino, amino may be acetylated to give acetylamino, etc.

The skilled person will also appreciate that various standard substituent or functional group interconversions and transformations within certain compounds of formula I will provide other compounds of formula I. For example, carbonyl may be reduced to hydroxy or alkylene, hydroxy may be acylated to give alkylcarbonyloxy, nitro may be reduced to amino, amido may be reduced to amino, amino may be sulfonated or acylated to give sulfonylamino or acylamino (respectively), and certain acyclic groups may be converted to certain heterocyclic groups under conditions known to those skilled in the art, for example as described in Comprehensive Heterocyclic Chemistry II, edited by A R Katritsky, C W Rees and EFV Scriven, 1^{st} Edition, Elsevier Science Ltd., Volumes 1-11 (1996).

The compounds of the invention may be isolated from their reaction mixtures using conventional techniques.

It will be appreciated by those skilled in the art that, in the process described above, the functional groups of intermediate compounds may be, or may need to be, protected by protecting groups.

Functional groups which it is desirable to protect include hydroxy, amino and carboxylic acid. Suitable protecting groups for hydroxy include trialkylsilyl and diarylalkylsilyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). Suitable protecting groups for amino include benzyl, tert-butyloxycarbonyl, 9-fluorenylmethoxy-carbonyl or benzyloxycarbonyl. Suitable protecting groups for amidino and guanidino include benzyloxycarbonyl. Suitable protecting groups for carboxylic acid include C₁₋₆ alkyl or benzyl esters.

The protection and deprotection of functional groups may take place before or after any of the reaction steps described hereinbefore.

Protecting groups may be removed in accordance with techniques which are well known to those skilled in the art and as described hereinafter.

The use of protecting groups is fully described in “Protective Groups in Organic Chemistry”, edited by J. W. F. McOmie, Plenum Press (1973), and “Protective Groups in Organic Synthesis”, 3_{rd} edition, T. W. Greene & P. G. M. Wutz, Wiley-Interscience (1999).

Persons skilled in the art will appreciate that, in order to obtain compounds of the invention in an alternative, and, on some occasions, more convenient, manner, the individual process steps mentioned herein may be performed in a different order, and/or the individual reactions may be performed at a different stage in the overall route (i.e. substituents may be added to and/or chemical transformations performed upon, different intermediates to those associated hereinbefore with a particular reaction).

This will depend inter alia on factors such as the nature of other functional groups present in a particular substrate, the availability of key intermediates and the protecting group strategy (if any) to be adopted. Clearly, the type of chemistry involved will influence the choice of reagent that is used in the said synthetic steps, the need, and type, of protecting groups that are employed, and the sequence for accomplishing the synthesis.

It will also be appreciated by those skilled in the art that, although certain protected derivatives of compounds of formula I, which may be made prior to a final deprotection stage, may not possess pharmacological activity as such, they may be administered parenterally or orally and thereafter metabolised in the body to form compounds of the invention which are pharmacologically active. Such derivatives may therefore be described as “prodrugs”. Moreover, certain compounds of formula I may act as prodrugs of other compounds of formula I.

All prodrugs of compounds of formula I are included within the scope of the invention.

Some of the intermediates referred to hereinbefore are novel. According to a further aspect of the invention there is thus provided: (a) a compound of formula II, as hereinbefore defined, or a protected derivative thereof; (b) a compound of formula VI, as hereinbefore defined, or a protected derivative thereof; (c) a compound of formula VII, as hereinbefore defined, or a protected derivative thereof; (d) a compound of formula IX, as hereinbefore defined, or a protected derivative thereof; (e) a compound of formula XI, as hereinbefore defined, or a protected derivative thereof; (f) a compound of formula XIII, as hereinbefore defined, or a protected derivative thereof; (g) a compound of formula XVII, as hereinbefore defined, or a protected derivative thereof; (h) a compound of formula XIX, as hereinbefore defined (provided that at least one of R^{3a} and R^{3b} represents —N(R³⁹)R^{38c}, wherein R³⁹ represents a structural fragment of formula Ia, as hereinbefore defined), or a protected derivative thereof, (i) a compound of formula XXII, as hereinbefore defined (provided that at least one of R^{3a} and R^{3b} represents —N(R³⁹)R^{38c}, wherein R³⁹ represents a structural fragment of formula Ia, as hereinbefore defined), or a protected derivative thereof; and (J) a compound of formula XXIII, as hereinbefore defined, or a protected derivative thereof.

Compounds of formula II that may be mentioned include those in which: when R^{3a} and R^{3b} independently represent H, C₁₋₄ alkyl, OH or N(R³⁹)R^{38c}; and R³⁹ represents H or C₁₋₆ alkyl; then R² does not represent:

(i) CN;

(ii) C₁₋₆ alkyl optionally substituted by OH, N(R³⁰)R³¹ or Het⁹; wherein R³⁰ and R³¹ independently represent H, C₁₋₆ alkyl or C₃₋₈ cycloalkyl); and Het⁹ represents an unsubstituted, saturated 3- to 8-membered heterocycle containing one nitrogen atom (via which atom the heterocyclic group is attached to the rest of the molecule);

(iii) C₁₋₆ n-alkyl, which alkyl group is optionally interrupted by O and is terminated by N(R³⁰)R³¹ or OR³²; wherein one of R³⁰ and R³¹ represents H or phenyl substituted in the meta- or para-position (relative to the point of attachment) by CO₂H or NH₂ and the other represents H or C₁₋₆ alkyl; and R³² represents H or phenyl substituted in the meta- or para-position (relative to the point of attachment) by CO₂H or NH₂;

(iv) —C(O)R¹⁶, wherein R¹⁶ represents H or phenyl substituted in the meta- or para-position (relative to the point of attachment) by CO₂H or NH₂;

(v) —S(O)₂R²², wherein R²² represents phenyl substituted in the meta- or para-position (relative to the point of attachment) by CO₂H or NH₂;

(vi) —S(O)₂N(R²³)R²⁴; wherein R²³ represents phenyl substituted in the beta- or para-position (relative to the point of attachment) by CO₂H or NH₂; and R²⁴ represents H or C₁₋₆ alkyl; and

(Vii) C₃₋₄ n-alkyl, which alkyl group is terminated by phenyl, which phenyl group is substituted in the meta- or para-position (relative to the point of attachment) by CO₂H or NH₂, and which alkyl group is interrupted at the β-position (relative to the point of attachment of the phenyl group) by O.

Compounds of formula II, IX and XI that may be mentioned include those in which:

when R^{3a} and R^{3b} independently represent H, C₁₋₄ alkyl, OH or N(R³⁹)R^{38c};

R³⁹ represents H or C₁₋₆ alkyl; and

R⁴ and R⁵ both represent H;

then R² does not represent CN, —C(O)R¹⁶, —S(O)₂₂, —S(O)R²N(R²³)R²⁴ or C₁₋₆ alkyl optionally substituted as defined hereinbefore in respect of R².

Further compounds of formulae II, IX and XI that may be mentioned include those in which:

R² represents Het⁸, —C(O)R¹⁶, —C(S)OR¹⁷, —C(S)N(R¹⁸)R¹⁹—[C(O)]₂N(R^{20a})R^{20b}, —[C(O)]₂OR²¹, —S(O)₂R²², —S(O)₂N(R²³)R²⁴, —C(═N—CN)N(R²⁵)R²⁶, —C(═N—CN)OR²⁷ or C₁₋₁₂ alkyl (which alkyl group is substituted and/or terminated by one or more substituents selected from —C(O)R²⁸, —C(O)N(R^{29a})R^{29b}, —N(R³⁰)R³¹, —OR³², —S(O)ᵣR³³ halo, —CN and nitro);

R¹⁶ represents Het¹⁰ or C₁₋₆ alkyl (which alkyl group is optionally substituted and/or terminated by one or more substituents selected from halo, —OH, —CN, —N(R³⁴)R³⁵, aryl and Het¹¹);

R²² represents Het¹⁴ or C₁₋₆ alkyl (which alkyl group is optionally substituted and/or terminated by one or more substituents selected from halo, —OH, —CN, Het¹⁵ and aryl);

R²³ represents H, C₁₋₆ alkyl, Het¹⁶, —C(O)R^{36a}, —C(O)OR^{36b} or —C(O)SR^{36b};

R³⁰ represents Het¹⁹, —C(O)R^{37a}, —C(O)OR^{37b} or —C(O)N(R^{37c})R^{37d};

R³² represents C₁₋₆ alkyl, Het²¹, —C(O)R^{37a}, —C(O)OR^{37b} or —C(O)N(R^{37c})R^{37d}.

Medical and Pharmaceutical Use

The compounds of the invention are useful because they possess pharmacological activity. They are therefore indicated as pharmaceuticals.

Thus, according to a further aspect of the invention there is provided the compounds of the invention for use as pharmaceuticals.

In particular, the compounds of the invention exhibit myocardial electrophysiological activity, for example as demonstrated in the test described below.

The compounds of the invention are thus expected to be useful in both the prophylaxis and the treatment of arrhythmias, and in particular atrial and ventricular arrhythmias.

The compounds of the invention are thus indicated in the treatment or prophylaxis of cardiac diseases, or in indications related to cardiac diseases, in which arrhythmias are believed to play a major role, including ischaemic heart disease, sudden heart attack, myocardial infarction, heart failure, cardiac surgery and thromboembolic events.

In the treatment of arrhythmias, compounds of the invention, have been found to selectively delay cardiac repolarization, thus prolonging the QT interval, and, in particular, to exhibit class III activity. Although compounds of the invention have been found to exhibit class III activity in particular, in the treatment of arrhythmias, their mode(s) of activity is/are not necessarily restricted to this class.

According to a further aspect of the invention, there is provided a method of treatment of an arrhythmia which method comprises administration of a therapeutically effective amount of a compound of the invention to a person suffering from, or susceptible to, such a condition.

Pharmaceutical Preparations

The compounds of the invention will normally be administered orally, subcutaneously, intravenously, intraarterially, transdermally, intranasally, by inhalation, or by any other parenteral route, in the form of pharmaceutical preparations comprising the active ingredient either as a free base, a pharmaceutically acceptable ion exchanger or a non-toxic organic or inorganic acid addition salt, in a pharmaceutically acceptable dosage form. Depending upon the disorder and patient to be treated, as well as the route of administration, the compositions may be administered at varying doses.

The compounds of the invention may also be combined with any other drugs useful in the treatment of arrhythmias and/or other cardiovascular disorders.

According to a further aspect of the invention there is thus provided a pharmaceutical formulation including a compound of the invention in admixture with a pharmaceutically acceptable adjuvant, diluent or carrier. Suitable daily doses of the compounds of the invention in therapeutic treatment of humans are about 0.005 to 10.0 mg/kg body weight at oral administration and about 0.005 to 5.0 mg/kg body weight at parenteral administration.

The compounds of the invention have the advantage that they are effective against cardiac arrhythmias.

Compounds of the invention may also have the advantage that they may be more efficacious than, be less toxic than, have a broader range of activity (including exhibiting any combination of class I, class II, class III and/or class IV activity (especially class I and/or class IV activity in addition to class III activity)) than, be more potent than, be longer acting than, produce fewer side effects (including a lower incidence of proarrhythmias such as torsades de pointes) than, be more easily absorbed than, or that they may have other useful pharmacological properties over, compounds known in the prior art.

Biological Tests

Test A

Primary Electrophysiological Effects In Anaesthetised Guinea Pigs

Guinea pigs weighing between 660 and 1100 g were used. The animals were housed for at least one week before the experiment and had free access to food and tap water during that period.

Anaesthesia was induced by an intraperitoneal injection of pentobarbital (40 to 50 mg/kg) and catheters were introduced into one carotid artery (for blood pressure recording and blood sampling) and into one jugular vein (for drug infusions). Needle electrodes were placed on the limbs for recording of ECGs (lead II). A thermistor was placed in the rectum and the animal was placed on a heating pad, set to a rectal temperature of between 37.5 and 38.5° C.

A tracheotomy was performed and the animal was artificially ventilated with room air by use of a small animal ventilator, set to keep blood gases within the normal range for the species. In order to reduce autonomic influences both vagi were cut in the neck, and 0.5 mg/kg of propranolol was given intravenously, 15 minutes before the start of the experiment.

The left ventricular epicardium was exposed by a left-sided thoracotomy, and a custom-designed suction electrode for recording of the monophasic action potential (MAP) was applied to the left ventricular free wall. The electrode was kept in position as long as an acceptable signal could be recorded, otherwise it was moved to a new position. A bipolar electrode for pacing was clipped to the left atrium. Pacing (2 ms duration, twice the diastolic threshold) was performed with a custom-made constant current stimulator. The heart was paced at a frequency just above the normal sinus rate during 1 minute every fifth minute throughout the study.

The blood pressure, the MAP signal and the lead II ECG were recorded on a Mingograph ink-jet recorder (Siemens-Elema, Sweden). All signals were collected (sampling frequency 1000 Hz) on a PC during the last 10 seconds of each pacing sequence and the last 10 seconds of the following minute of sinus rhythm. The signals were processed using a custom-made program developed for acquisition and analysis of physiological signals measured in experimental animals (see Axenborg and Hirsch, Comput. Methods Programs Biomed. 41, 55 (1993)).

The test procedure consisted of taking two basal control recordings, 5 minutes apart, during both pacing and sinus rhythm. After the second control recording, the first dose of the test substance was infused in a volume of 0.2 mL into the jugular vein catheter for 30 seconds. Three minutes later, pacing was started and a new recording was made. Five minutes after the previous dose, the next dose of test substance was administered. Six to ten consecutive doses were given during each experiment.

Data Analysis

Of the numerous variables measured in this analysis, three were selected as the most important for comparison and selection of active compounds. The three variables selected were the MAP duration at 75 percent repolarization during pacing, the atrio-ventricular (AV) conduction time (defined as the interval between the atrial pace pulse and the start of the ventricular MAP) during pacing, and the heart rate (defined as the RR interval during sinus rhythm). Systolic and diastolic blood pressure were measured, in order to judge the haemodynamic status of the anaesthetised animal. Further, the ECG was checked for arrhythmias and/or morphological changes.

The mean of the two control recordings was set to zero and the effects recorded after consecutive doses of test substance were expressed as percentage changes from this value. By plotting these percentage values against the cumulative dose administered before each recording, it was possible to construct dose-response curves. In this way, each experment generated three dose-response curves, one for MAP duration, one for AV-conduction time and one for the sinus frequency (RR interval). A mean curve of all experiments performed with a test substance was calculated, and potency values were derived from the mean curve. All dose-response curves in these experiments were constructed by linear connection of the data points obtained. The cumulative dose prolonging the MAP duration by 10% from the baseline was used as an index to assess the class III electrophysiological potency of the agent under investigation (D₁₀).

Test B

Glucocorticoid-Treated Mouse Fibroblasts as a Model to Detect Blockers of the Delayed Rectifier K Current

IC50 for K channel blockade was determined using a microtitre plate based screen method, based on membrane potential changes of glucocorticoid-treated mouse fibroblasts. The membrane potential of glucocorticoid-treated mouse fibroblasts was measured using fluorescence of the bisoxonol dye DiBac₄₍₃₎, which could be reliably detected using a fluorescence laser imaging plate reader (FLIPR). Expression of a delayed rectifier potassium channel was induced in mouse fibroblasts by 24 hours exposure to the glucocorticoide dexamehasone (5 μM). Blockade of these potassium channels depolarised the fibroblasts, resulting in increased fluorescence of DiBac₄₍₃₎.

Mouse ltk fibroblasts (L-cells) were purchased from American Type Culture Collection (ATCC, Manassa, Va.), and were cultured in Dulbeccos modified eagle medium supplemented with fetal calf serum (5% vol/vol), penicillin (500 units/mL), streptomycin (500 μg/mL) and L-alanine-L-glutamine (0.862 mg/mL). The cells were passaged every 3-4 days using trypsin (0.5 mg/mL in calcium-free phosphate buffered saline, Gibco BRL). Three days prior to experiments, cell-suspension was pipetted out into clear-bottom, black plastic, 96-well plates (Costar) at 25 000 cells/well.

The fluorescence probe DiBac₄₍₃₎ (DiBac Molecular probes) was used to measure membrane potential. DiBac₄₍₃₎ maximally absorbs at 488 nM and emits at 513 nM. DiBac₄₍₃₎ is a bisoxonol, and thus is negatively charged at pH 7. Due to its negative charge, the distribution of DiBac₄₍₃₎ across the membrane is dependent upon the transmembrane potential: if the cell depolarizes (i.e. the cell interior becomes less negative relative to cell exterior), the DiBac₄₍₃₎ concentration inside the cell increases, due to electrostatic forces. Once inside the cell, DiBac₄₍₃₎ molecules can bind to lipids and proteins, which causes an increase in fluorescence emission. Thus, a depolarization will be reflected by an increase in DiBac₄₍₃₎ fluorescence. The change in DiBac₄₍₃₎ fluorescence was detected by a FLIPR.

Prior to each experiment, the cells were washed 4 times in phosphate-buffered saline (PBS) to remove all culture media. The cells were then treated with 5 μM DiBac₄₍₃₎ (in 180 μL of PBS) at 35° C. Once a stable fluorescence was reached (usually after 10 min), 20 μL of the test substance was added, using FLIPR's internal 96 well pipetting system. Fluorescence measurements were then taken every 20 sec for a further 10 min. All experiments were carried out at 35° C., due to the high temperature sensitivity of both delayed rectifier potassium channel conductance and DiBac₄₍₃₎ fluorescence. Test substances were prepared in a second 96 well plate, in PBS containing 5 μM DiBac₄₍₃₎. The concentration of substance prepared was 10 times that of the desired concentration in the experiment as an additional 1:10 dilution occurred during addition of substance during the experiment. Dofetilide (10 μM) was used as a positive control, i.e. to determine the maximum increase in fluorescence.

Curve-fitting, used to determine the IC50 values, was performed with the Graphpad Prism program (Graphpad Software Inc., San Diego, Calif.).

Test C

Metabolic Stability of Test Compounds

An in vitro screen was set up to determine the metabolic stability of the compounds of the invention.

The hepatic S-9 fraction from dog, man, rabbit and rat with NADPH as co-factor was used. The assay conditions were as follows: S-9 (3 mg/mL), NADPH (0.83 mM), Tris-HCl buffer (50 mM) at pH 7.4 and 10 μM of test compound.

The reaction was started by addition of test compound and terminated after 0, 1, 5, 15 and 30 minutes by raising the pH in the sample to above 10 (NaOH; 1 mM). After solvent extraction, the concentration of test compound was measured against an internal standard by LC (fluorescence/UV detection).

The percentage of test compound remaining after 30 minutes (and thus t_{1/2}) was calculated and used as a measure for metabolic stability.

The invention is illustrated by way of the following examples.

### EXAMPLES

General Experimental Procedures

Mass spectra were recorded on one of the following instruments: a Finnigan MAT TSQ 700 triple quadrupole mass spectrometer equipped with an electrospray interface (FAB-MS); a Perkin-Elmer SciX API 150ex spectrometer; a VG Quattro II triple quadrupole; a VG Platform II single quadrupole; or a Micromass Platform LCZ single quadrupole mass spectrometer (the latter three instruments were equipped with a pneumatically assisted electrospray interface (LC-MS)). ¹H NMR and ¹³C NMR measurements were performed on a BRUKER ACP 300 and Varian 300, 400 and 500 spectrometers, operating at ¹H frequencies of 300, 400 and 500 MHz respectively, and at ¹³C frequencies of 75.5, 100.6 and 125.7 MHz respectively. Alternatively, ¹³C NMR measurements were performed on a BRUKER ACE 200 spectrometer at a frequency of 50.3 MHz.

Rotamers may or may not be denoted in spectra depending upon ease of interpretation of spectra. Unless otherwise stated, chemical shifts are given in ppm with the solvent as internal standard.

### EXAMPLE 1

tert-Butyl 2-{7-[3-(4-cyanophenoxy)-2-hydroxypropyl]-3,7-diazabicyclo-[3.3.1]non-3-yl}ethylcarbamate

(i) tert-Butyl 2-bromoethylcarbamate

A mixture of 2-bromoethylamine hydrobromide (10.0 g, 0.049 mol), NaOH (1.84 g, 0.046 mol), water (50 mL) and THF (200 mL) was cooled to 0° C. Di-tert-butyl dicarbonate (10.1 g, 0.046 mol) was added slowly to the mixture, which was then stirred at rt overnight. The mixture was concentrated in vacuo and the residue dissolved in DCM. This organic solution was washed with water and purified by chromatography on silica, eluting with DCM, to give 5.6 g (50%) of the title compound.

(ii) 4-[3-(3,7-Diazabicyclo[3.3.1]non-3-yl)-2-hydroxypropoxy]benzonitrile

HCl-saturated EtOAc (600 mL) was added to a solution of tert-butyl 7-[3-(4-cyanophenoxy)-2-hydroxypropyl]-3,7-diazabicyclo[3.3.1]nonane-3-carboxylate (62 g; see Example 2 of international patent application No. PCT/SE98/02276) in EtOAc (600 mL) and the mixture was stirred at rt. for 4 h. The solvent was removed under reduced pressure, the residue was dissolved in MeCN (1.3 L) and K₂CO₃ (100 g) was added. The suspension was stirred for 12 h and filtered. Concentration of the filtrate gave the title compound in a 90% yield.

¹³C NMR (CDCl₃): δ28.9, 29.2, 32.3, 50.9, 57.7, 60.8, 62.1, 66.0, 71.2, 104.0, 115.3, 119.1, 133.9, 162.1.

(iii) tert-Butyl 2-{7-[3-(4-cyanophenoxy)-2-hydroxypropyl]-3,7-diaza-bicyclo [3.3.1]non-3-yl}ethylcarbamate

4-[3-(3,7-Diazabicyclo[3.3.1]non-3-yl)-2-hydroxypropoxy]benzonitrile (see step (ii) above; 7.7 g, 25.6 mmol) and tert-butyl 2-bromoethylcarbamate (see step (i) above; 5.7 g, 2.56 mmol) and K₂CO₃ (3.5 g, 2.56 mmol) were mixed in CH₃CN (50 mL) and stirred at 60° C. for 60 h. The reaction mixture was filtered and evaporated. The residue was purified using column chromatography (DCM: 10-20% MeOH saturated with NH₃ (g)), to yield 8 g (71%) of the title compound.

¹³C NMR (CDCl₃): δ28.4, 29.6, 30.3, 32.0, 36.9, 54.8, 58.4, 58.6, 59.5, 64.8, 71.1, 78.8, 104.1, 115.3, 119.2, 133.9, 156.4, 162.2.

F [symbol] B-MS (M+1)⁺=445 (m/z)

### EXAMPLE 2

4-{3-[7-(3,3-Dimethyl-2-oxobutyl)-3,7-diazabicyclo[3.3.1]non-3-yl]-2-hydroxypropoxy}benzonitrile

4-[3-(3,7-Diazabicyclo[3.3.1]non-3-yl)-2-hydroxypropoxy]benzonitrile (see Example 1(ii) above; 0.6 g, 2.0 mmol) and 1-chloropinacolone (0.27 g, 2.0 mmol) and K₂CO₃ (0.27 g, 20 mmol) were mixed in CH₃CN and stirred at 60° C. for 1 h, and then at r.t. overnight. The reaction mixture was filtered and evaporated, giving 0.7 g (90%) of the title compound.

¹³C NMR (CDCl₃): δ26.2, 30.0, 30.6, 31.9, 43.5, 55.1, 57.3, 57.6, 59.2, 59.8, 61.7, 64.8, 71.1, 103.9, 115.3, 119.2, 133.9, 162.3, 212.2.

FAB-MS (M+1)⁺=400 (m/z)

### EXAMPLE 3

4-{3-[7-(2-Ethyl-2H-1,2,3,4-tetrazol-5-yl)-3,7-diazabicyclo[3.3.1]non-3-yl]-2-hydroxypropoxy}benzonitrile

(i) 3,7-Dibenzyl-3,7-diazabicyclo[3.3.1]nonane-9-one

The sub-title compound was prepared according to the procedure described in J. Org. Chem., 41(9), 1976, pp. 1593-1597.

(ii) 3,7-Dibenzyl-3,7-diazabicyclo[3.3.1]nonane

The sub-title compound was prepared according to the procedure described in J. Org. Chem., 41(9), 1976, pp. 1593-1597, using 3,7-dibenzyl-3,7-diazabicyclo [3.3.1]nonane-9-one (see step (i) above) in place of N-benzyl-N ′-methylbispidone.

(iii) 3-Benzyl-3,7-diazabicyclo[3.3.1]nonane

A solution of 3,7-dibenzyl-3,7-diazabicyclo[3.3.1]nonane (see step (ii) above; 97 g, 6.4 mmol) in aqueous ethanol (95%) was hydrogenated over 5% Pd/C at 1 atm. until tic indicated that the reaction was complete. The catalyst was removed by filtration through a pad of Celite®, and the filtrate concentrated under reduced pressure to give the sub-title compound in quantitative yield.

¹³C NMR (CDCl₃): δ30.1, 33.4, 36.0, 52.5, 59.6, 64.3, 126.9, 128.3, 128.7, 138.8

(iv) 3-Benzyl-7-cyano-3,7-diazabicyclo[3.3.1]nonane

3-Benzyl-3,7-diazabicyclo[3.3.1]nonane (see step (iii) above; 20 g, 92 mmol) was dissolved in ether (120 mL). Cyanogen bromide (9.8 g, 92 mmol) dissolved in ether (80 mL) was added dropwise at 0° C. The mixture was stirred at 0° C. for 15 minutes, and then at r.t. overnight, after which a white precipitate formed. The ether was evaporated. Water and a saturated Na₂CO₃ (aq) solution were added. The mixture was extracted with ether. The ether layer was separated and dried (MgSO₄), giving 20.3 g (91%) of the sub-title compound.

(v) 3-Benzyl-7-(2H-1,2,3,4-tetrazol-5-yl)-3,7-diazabicyclo[3.3.1]nonane, ammonium salt

A mixture of 3-benzyl-7-cyano-3,7-diazabicyclo[3.3.1]nonane (see step (iv) above; 10.2 g, 42 mmol), NaN₃ (2.92 g, 45 mmol), NH₄Cl (2.41 g, 45 mmol) and DMF (50 mL) was stirred at 100° C. for 22 h. DMF was evaporated, toluene was added and evaporated, which resulted in 12 g of an orange-coloured powder. The product was purified by preparative reversed phase HPLC, giving 5.8 g (46%) of the sub-title compound.

(vi) 3-Benzyl-7-(2-ethyl-2H-1,2,3,4-tetrazol-5-yl)-3,7-diazabicyclo[3.3.1]-nonane

A mixture of 3-benzyl-7-(2H-1,2,3,4-tetrazol-5-yl)-3,7-diazabicyclo-[3.3.1]nonane, ammonium salt (see step (v) above; 4 g, 13 mmol), ethyl iodide (2.20 mL, 26 mmol) and NaOH (0.62 g, 15.6 mmol) was refluxed for 2 h. The solvent was evaporated and the residue purified by flash chromatography (hexane:ethyl acetate (1:1), MeOH (NH3) 0-32%), giving 1.5 g (37%) of the sub-title compound.

(vii) 3-(2-Ethyl-2H-1,2,3,4-tetrazol-5-yl)-3,7-diazabicyclo[3.3.1]nonane

3-Benzyl-7-(2-ethyl-2H-1,2,3,4-tetrazol-5-yl)-3,7-diazabicyclo[3.3.1]-nonane (see step (vi) above; 0.5 g, 1.6 mmol) dissolved in ethanol (5 mL of 95%) was hydrogenated over 5% Pd/C at 1 atm overnight. The catalyst was filtered over a pad of Celite®, and the residue was evaporated to give 0.2 g (56%) of the sub-title compound.

(viii) 4-{3-[7-(2-Ethyl-2H-1,2,3,4-tetrazol-5-yl)-3,7-diazabicyclo[3.3.1]-non-3-yl]-2-hydroxypropoxy}benzonitrile

4-(2-Oxiranylmethoxy)benzonitrile (see international patent application WO 99/31100; 0.18 g, 1 mmol) and 3-(2-ethyl-2H-1,2,3,4-tetrazol-5-yl)-3,7-diazabicyclo[3.3.1]nonane (see step (vii) above; 0.2 g, 0.9 mmol) were mixed in isopropanol:H₂O (1.1 mL of 10:1) and the mixture was stirred at 60° C. overnight. The solvent was evaporated and the residue was purified by chromatography (hexane:ethyl acetate (1:1), MeOH (NH₃) 0-32%), giving 0.28 g (77%) of the title compound.

¹³C NMR (CD₃CN): δ14.4, 29.7, 30.0, 31.2, 48.6, 51.2, 51.3, 58.6, 60.8, 61.0, 66.3, 71.9, 104.3, 116.2, 118.2, 119.9, 134.9, 163.3, 170.1.

### EXAMPLE 4

4-{2-Hydroxy-3-[7-(1,3-thiazol-2-yl)-3,7-diazabicyclo[3.3.1]non-3-yl]-propoxy}benzonitrile

4-[3-(3,7-Diazabicyclo[3.3.1]non-3-yl)-2-hydroxypropoxy]benzonitrile (see Example 1(ii) above; 1 g, 3.3 mmol) 2-bromothiazole (0.54 g, 3.3 mmol) and K₂CO₃ (0.91 g, 6.5 mmol) were mixed in DMF (15 mL). The mixture was stirred overnight at 60° C. The solvent was evaporated, toluene was added and then evaporated. The residue was dissolved in ethyl acetate and washed with NaOH solution (2 M). The organic layer was separated and dried (Na₂SO₄). The residue was purified by chromatography (hexane:ethyl acetate (1:1)). Yield: 0.57 g (45%).

¹³C NMR (CDCl₃): δ29.1, 29.4, 30.5, 52.8, 53.0, 57.9, 60.7, 65.3, 70.4, 104.1, 105.6, 115.3, 119.2, 133.9, 139.7, 162.0, 171.2.

### EXAMPLE 5

N′-Cyano-7-[3-(4-cyanophenoxy)-2-hydroxypropyl]-N-(3,4,5-trimethoxy-benzyl) -3,7-diazabicyclo[3.3.1]nonane-3-carboximidamide

(i) Phenyl N′-cyano-N-(3,4,5-trimethoxybenzyl)carbamimidoate

3,4,5-Trimethoxybenzylamine (1.08 mL, 6.3 mmol) was dissolved in isopropanol (10 mL). The mixture was cooled to 0° C. before diphenyl cyanocarbonimidoate (1.5 g, 6.3 mmol) was added in portions. The reaction mixture was allowed to reach r.t. and then stirred overnight at that temperature. The precipitate that formed was filtered off and was then purified by chromatography DCM:MeOH (gradient 100:0 to 99:1). Yield: 1.37 g (63.5%)

(ii) N′-Cyano-7-[3-(4-cyanophenoxy)-2-hydroxypropyl]-N-(3,4,5-tri-methoxybenzyl) -3,7-diazabicyclo[3.3.1]nonane-3-carboximidamide

4-[3-(3,7-Diazabicyclo[3.3.1]non-3-yl)-2-hydroxypropoxy]benzonitrile (see Example 1(ii) above; 1 g, 3.3 mmol) and phenyl N′-cyano-N-(3,4,5-trimethoxybenzyl) carbamimidoate (see step (i) above; 1.13 g, 3.3 mmol) were mixed with isopropanol (15 mL) and then stirred at reflux for 3 h. The mixture was cooled to r.t. and the product that formed was filtered off. The product was purified by chromatography (DCM:MeOH (gradient 0 to 1%), giving 1.67 g (92%) of the title compound.

¹³C NMR (CDCl₃): δ29.0, 29.3, 31.2, 47.9, 50.9, 51.3, 56.1, 57.1, 60.0, 60.7, 61.3, 65.6, 70.6, 103.9, 105.1, 115.2, 118.3, 119.1, 133.2, 133.9, 137.4, 153.3, 160.2, 161.9, 176.3, 176.4.

### EXAMPLE 6

4-{3-Amino-4-[7-(butylsulfonyl)-3,7-diazabicyclo[3.3.1]non-3-yl]butoxy}-benzonitrile

(i) 4-(3-Butenyloxy)benzonitrile

4-Cyanophenol (30 g, 250 mmol) was mixed with K₂CO₃ (72.5 g, 525 mmol) and stirred for 60 min. 4-Bromo-1-butene (50 g, 370 mmol) was added dropwise, and then the reaction mixture was stirred at 60° C. overnight. The solids were filtered of and then the solvents were evaporated. The residue was dissolved in DCM and washed with 1 N NaOH. The organic layer was separated, dried (Na₂SO₄) and evaporated, giving 37 g (58%) of the sub-title compound.

(ii) 4-[2-(2-Oxiranyl)ethoxy]benzonitrile

4-(3-Butenyloxy)benzonitrile (see step (i) above; 37 g, 0.21 mol) was mixed with mCPBA (61.6 g, 0.25 mol) and DCM (700 mL) and stirred at r.t. for 4 h. The reaction mixture was filtered and 2 mL of DMSO was added to destroy the excess mCPBA. The mixture was washed with NaHCO₃, then separated, dried and evaporated to give 38.7 g (97%) of the sub-title compound.

(iii) 4-(4-Amino-3-hydroxybutoxy)benzonitrile

4-[2-(2-Oxiranyl)ethoxy]benzonitrile (see step (ii) above; 38.5 g, 204 mmol) was mixed with aqueous NH₃ (1200 mL, conc.) and isopropanol (450 mL). The mixture was stirred at r.t. for 24 h. The solid (by-product) was filtered off, and the solvents were evaporated, giving 39.1 g (93%) of the sub-title compound.

(iv) tert-Butyl 4-(4-cyanophenoxy)-2-hydroxybutylcarbamate

4-(4-Amino-3-hydroxybutoxy)benzonitrile (see step (iii) above; 34.3 g, 166 mmol) was dissolved in THF:H₂O (600 mL of 8:2). Di-tert-butyl dicarbonate (36.3 g, 166 mmol) was added at 0° C. The mixture was then stirred at r.t. overnight before being evaporated to give 50 g (100%) of the sub-title compound (which was used in the next step without further purification).

(v) 1-{[(tert-Butoxycarbonyl)amino]methyl}-3-(4-cyanophenoxy)propyl methanesulfonate

tert-Butyl 4-(4-cyanophenoxy)-2-hydroxybutylcarbamate (see step (iv) above; 38.1 g, 120 mmol) and 4-(dimethylamino)pyridine (10 mol%) were dissolved in pyridine (200 mL). The mixture was cooled to 0° C. Methanesulfonyl chloride (10.7 mL, 0.136 mol) was then added dropwise at 0° C. The mixture was allowed to reach r.t. before the pyridine was evaporated. DCM was added and the solution was washed with 2 M HCl and water before being dried and evaporated. The compound was purified by chromatography on silica, eluting with DCM (5% ethyl acetate), to give 27 g of the sub-title compound.

(vi) tert-Butyl 2-[2-(4-cyanophenoxy)ethyl]-1-aziridinecarboxylate

1-{[(tert-Butoxycarbonyl)amino]methyl}-3-(4-cyanophenoxy)propyl methanesulfonate (see step (v) above; 25.3 g, 0.066 mol) was mixed with tetrabutylammonium hydrogen sulphate (2.7 g; 7.8 mmol) and DCM (170 mL). The mixture was cooled to 0° C. and NaOH (50% (aq)) was added slowly. The mixture was then allowed to reach r.t. before water and DCM were added. The organic layer was separated, washed with water, dried and then evaporated to give the sub-title compound. Yield: 19 g (99%). The product was used in the next step without further purification.

(vii) 3-Benzyl-7-(butylsulfonyl)-3,7-diazabicyclo[3.3.1]nonane

Butanesulfonyl chloride was added dropwise at 0° C. to a mixture of 3-benzyl-3,7-diazabicyclo[3.3.1]nonane (see Example 3(iii) above; 13 g, 60 mmol), K₂CO₃ (60 mmol) and MeCN (100 mL). The mixture was allowed to reach r.t. and was then stirred overnight at r.t. The reaction mixture was filtered through a plug of silica, which was then eluted with ethyl acetate to give 15 g (75%) of the sub-title compound.

(viii) 3-(Butylsulfonyl)-3,7-diazabicyclo[3.3.1]nonane

3-Benzyl-7-(butylsulfonyl)-3,7-diazabicyclo[3.3.1]nonane (see step (vii) above; 12.7 g, 38 mmol) was dissolved in ethanol (150 mL of 95%) and hydrogenated over 5% Pd/C at 1 atm. overnight. TLC analysis showed that no reaction had occurred. The catalyst was filtered off and new catalyst (5% Pd/C) was added, together with H₂O (10 mL) and acetic acid (2 mL). The mixture was then hydrogenated at 1 atm. overnight. The catalyst was filtered off, 2 N NaOH was added and the mixture was extracted with toluene. Evaporation of the toluene solution gave 8 g (85%) of the sub-title compound.

(ix) tert-Butyl 1-{[7-(butylsulfonyl)-3,7-diazabicyclo[3.3.1]non-3-yl]-methyl }-3-(4-cyanophenoxy)propylcarbamate

tert-Butyl 2-[2-(4-cyanophenoxy)ethyl]-1-aziridinecarboxylate (see step (vi) above; 1 g, 3.5 mmol) was mixed with 3-(butylsulfonyl)-3,7-diazabicyclo [3.3.1]nonane (see step (viii) above; 0.85 g, 3.5 mmol) in isopropanol (25 mL). The mixture was stirred at 60° C. overnight. Evaporation and purification of the residue on silica (DCM, 2% MeOH) gave 1.5 g (81%) of the sub-title compound.

¹H NMR (CDCl₃): δ1.44 (3H, t), 1.91 (9H, s), 2.08 (2H, m), 2.1 (2H, m), 2.2 (2H, m), 2.4 (2H, s (broad)), 2.6 (2H, m), 2.8 (2H, d), 2.9 (2H, dd), 3.3-3.6 (6H, m), 4.3 (3H, m), 4.6 (2H, m), 5.7 (1H, s (broad)), 7.4 (2H, dd), 8.0 (2H, dd).

(x) 4-{3-Amino-4-[7-(butylsulfonyl)-3,7-diazabicyclo[3.3.1]non-3-yl]-butoxy }benzontrile

tert-Butyl 1-{[7-(butylsulfonyl)-3,7-diazabicyclo[3.3.1]non-3-yl]methyl}-3-(4-cyanophenoxy)propylcarbamate (see step (ix) above; 0.9 g, 1.7 mmol) was dissolved in ethyl acetate (20 mL). Ethyl acetate saturated with HCl (g) (50 mL) was added at 0° C. The resulting mixture was allowed to reach r.t. for 2 h. The solvent was evaporated and the resulting residue was dissolved in water to give a solution that was freeze-dried. This gave the dihydrochloride salt of the title compound.

ES-MS (M+H)⁺=435 (m/z)

### EXAMPLE 7

The compounds listed below were prepared either in accordance with, or analogously to, methods described herein, or were otherwise prepared according to the following procedure: The appropriate 3- or 7-unsubstituted bispidine (dissolved in CHCl₃) was reacted with 2 eq. of the appropriate electrophile (dissolved in CH₃CN) in the presence of a base (2.5 eq. of K₂CO₃). The reaction mixtures were warmed to 50-100° C. When the reaction was ready (as determined by mass spectral analysis), the inorganic salts were filtered off and the reaction mixture was added to an ion exchange solid phase extraction plug (CBA). The plug was washed with CHCl₃ and the product was finally eluted with CHCl₃:MeOH:Et₃N (8:1:1). The products were analysed by HPLC and MS. Compounds with a purity less then 90% were purified by preparative HPLC.

Mass spectra of the compounds, where recorded, are in brackets:

4-(3-{7-[3-(4-cyanophenoxy)-2-hydroxypropyl]-3,7-diazabicyclo[3.3.1]-non-3-yl}-2-hydroxypropoxy)benzonitrile;

4-(3-{7-[3-(4-cyanophenoxy)-2-hydroxypropyl]-9-(1,2-ethylenedioxy)-3,7-diazabicyclo[3.3.1]non-3-yl}-2-hydroxypropoxy)benzonitrile;

4-{3-[7-[3-(4-cyanophenoxy)-2-hydroxypropyl]-9,9-bis(propylsulfanyl)-3,7-diazabicyclo[3.3.1]non-3-yl]-2-hydroxypropoxy}benzonitrile;

3,7-bis(4-nitrophenethyl)-3,7-diazabicyclo[3.3.1]nonane;

4-(3-{7-[3-(4-cyanophenoxy)-2-hydroxypropyl]-9,9-tetramethylene-3,7-diazabicyclo[3.3.1]non-3-yl}-2-hydroxypropoxy)benzonitrile;

4-{2-[7-(4-cyanophenethyl)-3,7-diazabicyclo[3.3.1]non-3-yl]ethyl}benzonitrile;

N-{4-[(7-{4-[(methylsulfonyl)amino]benzyl}-3,7-diazabicyclo[3.3.1]non-3-yl)methyl]phenyl}methanesulfonamide;

4-cyano-N-[2-(7-{2-[(4-cyanobenzoyl)amino]ethyl}-3,7-diazabicyclo-[3.3.1]non-3-yl)ethyl]benzamide;

4-(2-{7-[2-(4-cyanophenyl)-2-hydroxyethyl]-3,7-diazabicyclo[3.3.1]non-3-yl}-1-hydroxyethyl)benzonitrile;

4-{3-[(3,7-dibenzyl-3,7-diazabicyclo[3.3.1]non-9-yl)(methyl)amino]-2-hydroxypropoxy}benzonitrile;

2-[(7-{[6-cyano-4-(methylsulfonyl)-3,4-dihydro-2H-1,4-benzoxazin-2-yl]-methyl}-3,7-diazabicyclo[3.3.1]non-3-yl)methyl]-4-(methylsulfonyl)-3,4-dihydro-2H-1,4-benzoxazine-6-carbonitrile;

4-[2-hydroxy-3-(7-methyl-3,7-diazabicyclo[3.3.1]non-3-yl)propoxy]benzonitrile;

2-[(7-methyl-3,7-diazabicyclo[3.3.1]non-3-yl)methyl]-4-(methylsulfonyl)-3,4-dihydro-2H-1,4-benzoxazine-6-carbonitrile;

4-[3-(7-benzyl-3,7-diazabicyclo[3.3.1]non-3-yl)-2-hydroxypropoxyl]benzonitrile;

4-{2-hydroxy-3-[7-(4-oxoheptyl)-3,7-diazabicyclo[3.3.1]non-3-yl]-propoxy}benzonitrile;

4-[((2R)-3-{7-[(2R)-3-(4-cyanophenoxy)-2-hydroxypropyl]-3,7-diazabicyclo[3.3.1]non-3-yl}-2-hydroxypropyl)oxy]benzonitrile;

4-[((2S)-3-{7-[(2S)-3-(4-cyanophenoxy)-2-hydroxypropyl]-3,7-diazabicyclo[3.3.1]non-3-yl}-2-hydroxypropyl)oxy]benzonitrile;

4-[3-(7-butyryl-3,7-diazabicyclo[3.3.1]non-3-yl)-2-hydroxypropoxy]-benzonitrile;

4-{3-[7-(ethylsulfonyl)-3,7-diazabicyclo[3.3.1]non-3-yl]-2-hydroxypropoxy}benzonitrile;

4-[((2S)-3-{7-[(2R)-3-(4-cyanophenoxy)-2-hydroxypropyl]-3,7-diazabicyclo[3.3.1]non-3-yl}-2-hydroxypropyl)oxy]benzonitrile;

4-{[(2S)-2-hydroxy-3-(7-propionyl-3,7-diazabicyclo[3.3.1]non-3-yl)-propyl]oxy}benzonitrile;

4-{[(2R)-2-hydroxy-3-(7-propionyl-3,7-diazabicyclo[3.3.1]non-3-yl)-propyl]oxy}benzonitrile;

2-{7-[(2S)-3-(4-cyanophenoxy)-2-hydroxypropyl]-3,7-diazabicyclo[3.3.1]-non-3-yl}-N-ethyl-2-oxoacetamide;

2-{7-[(2R)-3-(4-cyanophenoxy)-2-hydroxypropyl]-3,7-diazabicyclo[3.3.1]-non-3-yl}-N-ethyl-2-oxoacetamide;

tert-butyl(1S)-2-(7-benzyl-3,7-diazabicyclo[3.3.1]non-3-yl)-1-[(4-cyanophenoxy)methy]ethylcarbamate;

tert-butyl(1S)-2-(4-cyanophenoxy)-1-({7-[(ethylamino)carbothioyl]-3,7-diazabicyclo[3.3.1]non-3-yl}methyl)ethylcarbamate;

7-[(2S)-3-(4-cyanophenoxy)-2-hydroxypropyl]-N-ethyl-3,7-diazabicyclo-[3.3.1]nonane-3-carbothioamide;

tert-butyl(1S)-1-({7-[(2S)-3-(4-cyanophenoxy)-2-hydroxypropyl]-3,7-diazabicyclo[3.3.1]non-3-yl}carbonyl)-2-methylpropylcarbamate;

2-(4-cyanophenoxy)-1-{[7-(ethylsulfonyl)-3,7-diazabicyclo[3.3.1]non-3-yl]methyl}ethyltert-butylcarbamate;

4-[((2S)-3-{7-[(2S)-2-amino-3-methylbutanoyl]-3,7-diazabicyclo[3.3.1]-non-3-yl}-2-hydroxypropyl)oxy]benzonitrile;

4-{[(2S)-2-hydroxy-3-(7-{[(2S)-5-oxopyrrolidinyl]carbonyl}-3,7-diazabicyclo[3.3.1]non-3-yl)propyl]oxy}benzonitrile;

N-(5-cyano-2-{3-[7-(ethylsulfonyl)-3,7-diazabicyclo[3.3.1]non-3-yl]-2-hydroxypropoxy}phenyl)-N′-ethylurea;

N-(2-{2-amino-3-[7-(ethylsulfonyl)-3,7-diazabicyclo[3.3.1]non-3-yI]-propoxy}-5-cyanophenyl)-N′-ethylurea;

4-{3-[7-(3,3-dimethylbutanoyl)-3,7-diazabicyclo[3.3.1]non-3-yl]-2-hydroxypropoxy}benzonitrile;

N-(2-{7-[3-(4-cyanophenoxy)-2-hydroxypropyl]-3,7-diazabicyclo[3.3.1]-non-3-yl}-2-oxoethyl)acetamide;

tert-butyl(1S)-2-{7-[(2S)-3-(4-cyanophenoxy)-2-hydroxypropyl]-3,7-diazabicyclo[3.3.1]non-3-yl}-1-(4-methoxybenzyl)-2-oxoethylcarbamate;

4-[((2S)-3-{7-[(2S)-2-amino-3-(4-methoxyphenyl)propanoyl]-3,7-diazabicyclo[3.3.1]non-3-yl}-2-hydroxypropyl)oxy]benzonitrile;

2-{7-[3-(4-cyanophenoxy)-2-hydroxypropyl]-3,7-diazabicyclo[3.3.1]non-3-yl}-N-(2,6-dimethylphenyl)acetamide (m/z=463);

tert-butyl2-{7-[3-(4-cyanophenoxy)-2-hydroxypropyl]-3,7-diazabicyclo-[3.3.1]non-3-yl}-2-oxoethylcarbamate (m/z=344);

4-{3-[7-(2-aminoethyl)-3,7-diazabicyclo[3.3.1]non-3-yl]-2-hydroxypropoxy}benzonitrile (m/z=345);

N-(2-{7-[3-(4-cyanophenoxy)-2-hydroxypropyl]-3,7-diazabicyclo[3.3.1]-non-3-yl}ethyl)-4-nitrobenzamide (m/z=494);

4-amino-N-(2-{7-[3-(4-cyanophenoxy)-2-hydroxypropyl]-3,7-diazabicyclo-[3.3.1]non-3-yl}ethyl)benzamide (m/z=464);

N-(2-{7-[3-(4-cyanophenoxy)-2-hydroxypropyl]-3,7-diazabicyclo[3.3.1]-non-3-yl}ethyl)-4-[(methylsulfonyl)amino]benzamide (m/z=542);

4-(acetylamino)-N-(2-{7-[3-(4-cyanophenoxy)-2-hydroxypropyl]-3,7-diazabicyclo[3.3.1]non-3-yl}ethyl)benzamide (m/z=506);

2-[7-(2-{[4-(acetylamino)benzoyl]amino}ethyl)-3,7-diazabicyclo[3.3.1]-non-3-yl]-1-[(4-cyanophenoxy)methyl]ethylacetate (m/z=548);

4-(3-{7-[(3,5-dimethyl-4-isoxazolyl)carbonyl]-3,7-diazabicyclo[3.3.1]non-3-yl}-2-hydroxypropoxy)benzonitrile (m/z=425);

4-{2-hydroxy-3-[7-(2-isopropyl-2H-1,2,3,4-tetraazol-5-yl)-3,7-diazabicyclo[3.3.1]non-3-yl]propoxy}benzonitrile (m/z=412);

4-(2-hydroxy-3-{7-[(5-methyl-3-isoxazolyl)carbonyl]-3,7-diazabicyclo-[3.3.1]non-3-yl}propoxy)benzonitrile (m/z=411);

4-[3-(7-{[3-(tert-butyl)-1-methyl-1H-pyrazol-5-yl]carbonyl}-3,7-diazabicyclo[3.3.1]non-3-yl)-2-hydroxypropoxy]benzonitrile (m/z=466);

4-(2-hydroxy-3-{7-[(4-methyl-1,2,3-thiadiazol-5-yl)carbonyl]-3,7-diazabicyclo[3.3.1]non-3-yl}propoxy)benzonitrile (m/z=428);

4-[3-(7-cyano-3,7-diazabicyclo[3.3.1]non-3-yl)-2-hydroxypropoxy]benzonitrile (m/z=327);

2-{7-[3-(4-cyanophenoxy)-2-hydroxypropyl]-3,7-diazabicyclo[3.3.1]non-3-yl}-2-oxoacetamide (m/z=373);

N-(3-{7-[3-(4-cyanophenoxy)-2-hydroxypropyl]-3,7-diazabicyclo[3.3.1]-non-3-yl}propyl)-N-(3,4-dimethoxyphenyl)-4-nitrobenzamide (m/z=644);

4-{[7-(4-oxoheptyl)-3,7-diazabicyclo[3.3.1]non-3-yl]sulfonyl}benzonitrile (m/z=404);

4-{2-hydroxy-3-[7-(1-phenyl-1H-1,2,3,4-tetraazol-5-yl)-3,7-diazabicyclo-[3.3.1]non-3-yl]propoxy}benzonitrile (m/z=446);

4-{2-hydroxy-3-[7-(1-methyl-1H-1,2,3,4-tetraazol-5-yl)-3,7-diazabicyclo-[3.3.1]non-3-yl]propoxy}benzonitrile(m/z=384);

4-{2-hydroxy-3-[7-(2-methyl-2H-1,2,3,4-tetraazol-5-yl)-3,7-diazabicyclo-[3.3.1]non-3-yl]propoxy}benzonitrile (m/z=384);

N′-cyano-7-[3-(4-cyanophenoxy)-2-hydroxypropyl]-N-cyclopropyl-3,7-diazabicyclo[3.3.1]nonane-3-carboximidamide;

S-propyl{7-[3-(4-cyanophenoxy)-2-hydroxypropyl]-3,7-diazabicyclo-[3.3.1]non-3-yl}sulfonylcarbamothioate (m/z=483);

4-((E)-3-{7-[3-(4-cyanophenoxy)-2-hydroxypropyl]-3,7-diazabicyclo-[3.3.1]non-3-yl}-3-oxo-1-propenyl)benzonitrile(m/z=457);

ethyl{7-[3-(4-cyanophenoxy)-2-hydroxypropyl]-3,7-diazabicyclo[3.3.1]-non-3-yl}carbothioylcarbamate;

4-(2-hydroxy-3-{7-[(2-oxo-1,3-oxazolidin-4-yl)methyl]-3,7-diazabicyclo-[3.3.1]non-3-yl}propoxy)benzonitrile;

tert-butyl2-{7-[2-amino-3-(4-cyanophenoxy)propyl]-3,7-diazabicyclo-[3.3.1]non-3-yl}ethylcarbamate (m/z=444);

tert-butyl3-[({7-[3-(4-cyanophenoxy)-2-hydroxypropyl]-3,7-diazabicyclo-[3.3.1]non-3-yl}carbothioyl)amino]propanoate;

tert-butyl2-{7-[3-(4-cyanoanilino)propyl]-3,7-diazabicyclo[3.3.1]non-3-yl}ethylcarbamate(m/z=428);

4-(3-{7-[(E)-3-(3,4-dimethoxyphenyl)-2-propenoyl]-3,7-diazabicyclo-[3.3.1]non-3-yl}-2-hydroxypropoxy)benzonitrile;

4-(3-{7-[(E)-3-(4-fluorophenyl)-2-propenoyl]-3,7-diazabicyclo[3.3.1]non-3-yl}-2-hydroxypropoxy)benzonitrile (m/z=450);

2-{7-[3-(4-cyanophenoxy)-2-hydroxypropyl]-3,7-diazabicyclo[3.3.1]non-3-yl}-N-isopropylacetamide (m/z=401);

4-({(2S)-3-[7-(cyclopropylmethyl)-9,9-tetramethylen-3,7-diazabicyclo-[3.3.1]non-3-yl]-2-hydroxypropyl}oxy)benzonitrile (m/z=410);

phenylN-cyano-7-[3-(4-cyanophenoxy)-2-hydroxypropyl]-3,7-diazabicyclo[3.3.1]nonane-3-carboximidoate (m/z=446);

4-{[7-(3,4-dimethoxyphenethyl)-3,7-diazabicyclo[3.3.1]non-3-yl]-carbonyl}benzonitrile (m/z=420);

4-{3-[7-(3-amino-1H-1,2,4-triazol-5-yl)-3,7-diazabicyclo[3.3.1]non-3-yl]-2-hydroxypropoxy}benzonitrile (m/z=384);

N′-cyano-7-[3-(4-cyanophenoxy)-2-hydroxypropyl]-N-(2-hydroxyethyl)-3,7-diazabicyclo[3.3.1]nonane-3-carboximidamide (m/z=413);

tert-butyl3-{7-[3-(4-cyanophenoxy)-2-hydroxypropyl]-3,7-diazabicyclo-[3.3.1]non-3-yl}-1-methyl-3-oxopropylcarbamate;

N′-cyano-7-[3-(4-cyanophenoxy)-2-hydroxypropyl]-N-[(5-hydroxy-1,3,3-trimethylcyclohexyl)methyl]-3,7-diazabicyclo[3.3.1]nonane-3-carboximidamide (m/z=523);

4-[2-hydroxy-3-(7-{2-hydroxy-3-[(2-methyl-1-oxo-1,2-dihydro-4-isoquinolinyl)oxy]propyl}-3,7-diazabicyclo[3.3.1]non-3-yl)propoxy]benzonitrile;

4-(3-{7-[(3,4-dimethoxyphenyl)sulfonyl]-3,7-diazabicyclo[3.3.1]non-3-yl}-2-hydroxypropoxy)benzonitrile (m/z=502);

4-{3-[7-(benzylsulfonyl)-3,7-diazabicyclo[3.3.1]non-3-yl]-2-hydroxypropoxy}benzonitrile(m/z=456);

4-{3-[7-(butylsulfonyl)-3,7-diazabicyclo[3.3.1]non-3-yl]-2-hydroxypropoxy}benzonitrile (m/z=422);

7-[3-(4-cyanophenoxy)-2-hydroxypropyl]-N,N-dimethyl-3,7-diazabicyclo-[3.3.1]nonane-3-sulfonamide;

4-{3-[7-(3-aminobutanoyl)-3,7-diazabicyclo[3.3.1]non-3-yl]-2-hydroxypropoxy}benzonitrile;

4-{3-[7-(1,3-dimethyl-2,6-dioxo-1,2,3,6-tetrahydro-4-pynrmidinyl)-3,7-diazabicyclo[3.3.1]non-3-yl]-2-hydroxypropoxy}benzonitrile (m/z=440);

N′-cyano-7-[3-(4-cyanophenoxy)-2-hydroxypropyl]-N-(2-methoxyethyl)-3,7-diazabicyclo[3.3.1]nonane-3-carboximidamide (m/z=427);

4-{2-hydroxy-3-[7-(2,2,2-trifluoroacetyl)-3,7-diazabicyclo[3.3.1]non-3-yl]propoxy}benzonitrile (m/z=398);

4-{2-hydroxy-3-[7-(3,3,3-trifluoropropanoyl)-3,7-diazabicyclo[3.3.1]non-3-yl]propoxy}benzonitrile;

N′-cyano-7-[3-(4-cyanophenoxy)-2-hydroxypropyl]-N-[(1S)-1-(1-naphthyl)ethyl]-3,7-diazabicyclo[3.3.1]nonane-3-carboximidamide(m/z=523);

N′-cyano-7-[3-(4-cyanophenoxy)-2-hydroxypropyl]-N-isopropyl-N-methyl-3,7-diazabicyclo[3.3.1]nonane-3-carboximidamide (m/z=425);

N′-cyano-7-[3-(4-cyanophenoxy)-2-hydroxypropyl]-N-(3,4-dimethoxyphenethyl)-3,7-diazabicyclo[3.3.1]nonane-3-carboximidamide;

N′-cyano-7-[3-(4-cyanophenoxy)-2-hydroxypropyl]-N-(3,4-dimethoxyphenethyl)-N-methyl-3,7-diazabicyclo[3.3.1]nonane-3-carboximidamide (m/z=547);

4-(2-hydroxy-3-{7-[(3-methyl-8-quinolinyl)sulfonyl]-3,7-diazabicyclo-[3.3.1]non-3-yl}propoxy)benzonitrile (m/z=507);

4-(2-hydroxy-3-{7-[(1-methyl-1H-imidazol-4-yl)sulfonyl]-3,7-diazabicyclo[3.3.1]non-3-yl}propoxy)benzonitrile (m/z=446);

4-(2-hydroxy-3-{7-[(trifluoromethyl)sulfonyl]-3,7-diazabicyclo[3.3.1]non-3-yl}propoxy)benzonitrile (m/z=434);

4-{2-hydroxy-3-[7-(2-oxobutyl)-3,7-diazabicyclo[3.3.1]non-3-yl]-propoxy}benzonitrile (m/z=372);

4-{3-[7-(2-furoyl)-3,7-diazabicyclo[3.3.1]non-3-yl]-2-hydroxypropoxy}-benzonitrile (m/z=396);

4-[2-hydroxy-3-(7-{[5-(2-pyridinyl)-2-thienyl]sulfonyl}-3,7-diazabicyclo-[3.3.1]non-3-yl)propoxy]benzonitrile (m/z=525);

N-[4-(2-{7-[3-(4-cyanophenoxy)-2-hydroxypropyl]-3,7-diazabicyclo-[3.3.1]non-3-yl}acetyl)phenyl]methanesulfonamide (m/z=513);

4-[2-hydroxy-3-(7-{[2-(4-morpholinyl)ethyl]sulfonyl}-3,7-diazabicyclo-[3.3.1]non-3-yl)propoxy]benzonitrile;

4-{3-[7-(4-amino-6,7-dimethoxy-2-quinazolinyl)-3,7-diazabicyclo[3.3.1]-non-3-yl]-2-hydroxypropoxy}benzonitrile;

3,7-bis[3-(4-cyanophenoxy)-2-hydroxypropyl]-7-aza-3-azoniabicyclo-[3.3.1]nonan-3-olate (mn/z=493);

4-(3-{7-[(3-fluorophenyl)sulfonyl]-3,7-diazabicyclo[3.3.1]non-3-yl}-2-hydroxypropoxy)benzonitrile (m/z=460);

4-{3-[7-(3,4-dimethoxyphenethyl)-3,7-diazabicyclo[3.3.1]non-3-yl]-2-hydroxypropoxy}benzonitrile (m/z=466);

4-[4-(7-butyryl-3,7-diazabicyclo[3.3.1]non-3-yl)-1-(3,4-dimethoxyphenoxy)butyl]benzonitrile (m/z=506);

4-[4-[7-(butylsulfonyl)-3,7-diazabicyclo[3.3.1]non-3-yl]-1-(3,4-dimethoxyphenoxy)butyl]benzonitrile (m/z=556);

4-{1-(3,4-dimethoxyphenoxy)-4-[7-(3,3-dimethyl-2-oxobutyl)-3,7-diazabicyclo[3.3.1]non-3-yl]butyl}benzonitrile (m/z=534);

4-[4-[7-(3,4-dimethoxyphenethyl)-3,7-diazabicyclo[3.3.1]non-3-yl]-1-(3,4-dimethoxyphenoxy)butyl]benzonitrile (m/z=600);

4-[4-(7-butyryl-3,7-diazabicyclo[3.3.1]non-3-yl)butyl]benzonitrile (m/z=354);

4-{2-[7-(butylsulfonyl)-3,7-diazabicyclo[3.3.1]non-3-yl]ethoxy}benzonitrile (m/z=404);

4-{4-[7-(3,3-dimethyl-2-oxobutyl)-3,7-diazabicyclo[3.3.1]non-3-yl]butyl}-benzonitrile (m/z=382);

4-{4-[7-(3,4-dimethoxyphenethyl)-3,7-diazabicyclo[3.3.1]non-3-yl]butyl}-benzonitrile (m/z=448);

4-[2-(7-butyryl-3,7-diazabicyclo[3.3.1]non-3-yl)ethoxy]benzonitrile (m/z=342);

4-{2-[7-(3,3-dimethyl-2-oxobutyl)-3,7-diazabicyclo[3.3.1]non-3-yl]-ethoxy}benzonitrile (m/z=370);

4-{2-[7-(3,4-dimethoxyphenethyl)-3,7-diazabicyclo[3.3.1]non-3-yl]-ethoxy}benzonitrile (m/z=436);

O-ethyl 7-[3-(4-cyanophenoxy)-2-hydroxypropyl]-3,7-diazabicyclo[3.3.1]-nonane-3-carbothioate;

benzonitrile,4-[2-hydroxy-3-[7-[(tetrahydro-3-thienyl)sulfonyl]-3,7-diazabicyclo[3.3.1]non-3-yl]propoxy]-, S,S-dioxide;

4-({(2S)-2-amino-3-[7-(3,3-dimethyl-2-oxobutyl)-3,7-diazabicyclo[3.3.1]-non-3-yl]propyl}oxy)benzonitrile;

4-{2-[7-(1,3-thiazol-2-yl)-3,7-diazabicyclo[3.3.1]non-3-yl]ethoxy}benzonitrile (m/z=355);

4-{1-(3,4-dimethoxyphenoxy)-4-[7-(1,3-thiazol-2-yl)-3,7-diazabicyclo-[3.3.1]non-3-yl]butyl}benzonitrile (m/z=519);

4-({3-[7-(1,3-thiazol-2-yl)-3,7-diazabicyclo[3.3.1]non-3-yl]propyl}-sulfonyl)benzonitrile (m/z=417);

4-cyano-N-{3-[7-(1,3-thiazol-2-yl)-3,7-diazabicyclo[3.3.1]non-3-yl]-propyl}benzamide (m/z=396);

4-{3-[7-(cyclopropylmethyl)-3,7-diazabicyclo[3.3.1]non-3-yl]-2-hydroxypropoxy}benzonitrile (m/z=356);

4-{2-[7-(cyclopropylmethyl)-3,7-diazabicyclo[3.3.1]non-3-yl]ethoxy}-benzonitrile (m/z=326);

4-[4-[7-(cyclopropylmethyl)-3,7-diazabicyclo[3.3.1]non-3-yl]-1-(3,4-dimethoxyphenoxy)butyl]benzonitrile (m/z=490);

4-({3-[7-(cyclopropylmethyl)-3,7-diazabicyclo[3.3.1]non-3-yl]propyl}-amino)benzonitrile (m/z=339);

4-{3-[7-(cyclopropylmethyl)-3,7-diazabicyclo[3.3.1]non-3-yl]-2-hydroxypropoxy}-N,N-dimethylbenzenesulfonamide (m/z=438);

4-({3-[7-(3,3-dimethyl-2-oxobutyl)-3,7-diazabicyclo[3.3.1]non-3-yl]-propyl}amino)benzonitrile (m/z=383);

4-(4-(4-cyanophenyl)-1-{2-[7-(3,3-dimethyl-2-oxobutyl)-3,7-diazabicyclo-[3.3.1]non-3-yl]-2-oxoethyl}-1H-pyrazol-5-yl)benzonitrile (m/z=535);

4-{3-[7-(3,3-dimethyl-2-oxobutyl)-3,7-diazabicyclo[3.3.1]non-3-yl]-2-hydroxypropoxy}-N,N-dimethylbenzenesulfonamide (m/z=482);

4-(2-{7-[2-(2-methoxyethoxy)ethyl]-3,7-diazabicyclo[3.3.1]non-3-yl}-ethoxy)benzonitrile (m/z=374);

N-[2-(4-cyanophenoxy)-1-({7-[2-(2-methoxyethoxy)ethyl]-3,7-diazabicyclo[3.3.1]non-3-yl}methyl)ethyl]-N′-methylurea (m/z=460);

4-[(3-{7-[2-(2-methoxyethoxy)ethyl]-3,7-diazabicyclo[3.3.1]non-3-yl}-propyl)amino]benzonitrile (m/z=387);

4-[4-(4-cyanophenyl)-1-(2-{7-[2-(2-methoxyethoxy)ethyl]-3,7-diazabicyclo[3.3.1]non-3-yl}-2-oxoethyl)-1H-pyrazol-5-yl]benzonitrile (m/z=539);

4-{3-[7-(4-fluorobenzyl)-3,7-diazabicyclo[3.3.1]non-3-yl]-2-hydroxypropoxy}benzonitrile (m/z=410);

4-{2-[7-(4-fluorobenzyl)-3,7-diazabicyclo[3.3.1]non-3-yl]ethoxy}benzonitrile (m/z=380);

4-{1-(3,4-dimethoxyphenoxy)-4-[7-(4-fluorobenzyl)-3,7-diazabicyclo-[3.3.1]non-3-yl]butyl}benzonitrile (m/z=544);

4-({3-[7-(4-fluorobenzyl)-3,7-diazabicyclo[3.3.1]non-3-yl]propyl}amino)-benzonitrile (m/z=393);

4-({3-[7-(4-fluorobenzyl)-3,7-diazabicyclo[3.3.1]non-3-yl]propyl}-sulfonyl)benzonitrile (m/z=442);

4-cyano-N-{3-[7-(4-fluorobenzyl)-3,7-diazabicyclo[3.3.1]non-3-yl]-propyl}benzamide (m/z=421);

4-{3-[7-(4-fluorobenzyl)-3,7-diazabicyclo[3.3.1]non-3-yl]-2-hydroxypropoxy}-N,N-dimethylbenzenesulfonamide (m/z=492);

4-{2-hydroxy-3-[7-(isopropylsulfonyl)-3,7-diazabicycl[3.3.1]non-3-yl]-propoxy}benzonitrile;

4-{3-[7-(1-cyanoethyl)-3,7-diazabicyclo[3.3.1]non-3-yl]-2-hydroxypropoxy}benzonitrile;

4-{2-[7-(1-cyanoethyl)-3,7-diazabicyclo[3.3.1]non-3-yl]ethoxy}benzonitrile;

4-[4-[7-(1-cyanoethyl)-3,7-diazabicyclo[3.3.1]non-3-yl]-1-(3,4-dimethoxy -phenoxy)butyl]benzonitrile (m/z=489);

4-{3-[7-(2-cyanopropyl)-3,7-diazabicyclo[3.3.1]non-3-yl]-2-hydroxypropoxy}-N,N-dimethylbenzenesulfonamide (m/z=451);

4-({3-[7-(3,4-dimethoxyphenethyl)-3,7-diazabicyclo[3.3.1]non-3-yl]-propyl}amino)benzonitrile (m/z=449);

4-({3-[7-(3,4-dimethoxyphenethyl)-3,7-diazabicyclo[3.3.1]non-3-yl]-propyl}sulfonyl)benzonitrile (m/z=498);

4-(4-(4-cyanophenyl)-1-{2-[7-(3,4-dimethoxyphenethyl)-3,7-diazabicyclo-[3.3.1]non-3-yl]-2-oxoethyl}-1H-pyrazol-5-yl)benzonitrile (m/z=601);

4-cyano-N-{3-[7-(3,4-dimethoxyphenethyl)-3,7-diazabicyclo[3.3.1]non-3-yl]propyl}benzamide (m/z=477);

4-{1-(3,4-dimethoxyphenoxy)-4-[7-(4-oxoheptyl)-3,7-diazabicyclo[3.3.1]-non-3-yl]butyl}benzonitrile (m/z=548);

4-(4-(4-cyanophenyl)-1-{2-oxo-2-[7-(4-oxoheptyl)-3,7-diazabicyclo[3.3.1]-non-3-yl]ethyl}-1H-pyrazol-5-yl)benzonitrile (m/z=549);

4-cyano-N-{3-[7-(4-oxoheptyl)-3,7-diazabicyclo[3.3.1]non-3-yl]propyl}-benzamide (m/z=425);

4-(3-{7-[2-(2,3-dihydro-1,4-benzodioxin-6-yl)-2-oxoethyl]-3,7-diazabicyclo[3.3.1]non-3-yl}-2-hydroxypropoxy)benzonitrile (m/z=478);

4-[(3-{7-[2-(2,3-dihydro-1,4-benzodioxin-6-yl)-2-oxoethyl]-3,7-diazabicyclo[3.3.1]non-3-yl}propyl)amino]benzonitrile (m/z=461);

4-(3-{7-[3-(ethylsulfonyl)propyl]-3,7-diazabicyclo[3.3.1]non-3-yl}-2-hydroxypropoxy)benzonitrile (m/z=436);

4-(1-(3,4-dimethoxyphenoxy)-4-{7-[3-(ethylsulfonyl)propyl]-3,7-diazabicyclo[3.3.1]non-3-yl}butyl)benzonitrile (m/z=570);

N-{2-{7-[3-(4-acetyl-1-piperazinyl)propyl]-3,7-diazabicyclo[3.3.1]non-3-yl}-1-[(4-cyanophenoxy)methyl]ethyl}-N′-methylurea (m/z=526);

4-[1-(2-{7-[3-(4-acetyl-1-piperazinyl)propyl]-3,7-diazabicyclo[3.3.1]non-3-yl}-2-oxoethyl)-4-(4-cyanophenyl)-1H-pyrazol-5-yl]benzonitrile (m/z=605);

4-({3-[7-(1,3-thiazol-2-yl)-3,7-diazabicyclo[3.3.1]non-3-yl]propyl}amino)-benzonitrile (m/z=368);

4-{2-hydroxy-3-[7-(1,3-thiazol-2-yl)-3,7-diazabicyclo[3.3.1]non-3-yl]-propoxy}-N,N-dimethylbenzenesulfonamide (m/z=467);

N-(2-(4-cyanophenoxy)-1-{[7-(cyclopropylmethyl)-3,7-diazabicyclo-[3.3.1]non-3-yl]methyl}ethyl)-N′-methylurea (m/z=412);

4-({3-[7-(cyclopropylmethyl)-3,7-diazabicyclo[3.3.1]non-3-yl]propyl}-sulfonyl)benzonitrile (m/z=388);

4-(2-{7-[3-(4-cyanophenoxy)-2-hydroxypropyl]-3,7-diazabicyclo[3.3.1]-non-3-yl}acetyl)benzonitrile;

N-(2-(4-cyanophenoxy)-1-{[7-(3,3-dimethyl-2-oxobutyl)-3,7-diazabicyclo-[3.3.1]non-3-yl]methyl}ethyl)-N′-methylurea (m/z=456);

4-({3-[7-(3,3-dimethyl-2-oxobutyl)-3,7-diazabicyclo[3.3.1]non-3-yl]-propyl}sulfonyl)benzonitrile (m/z=432);

4-cyano-N-{3-[7-(3,3-dimethyl-2-oxobutyl)-3,7-diazabicyclo[3.3.1]non-3-yl]propyl}benzamide (m/z=411);

4-(2-hydroxy-3-{7-[2-(2-methoxyethoxy)ethyl]-3,7-diazabicyclo[3.3.1]-non-3-yl}propoxy)benzonitrile (m/z=404);

4-(1-(3,4-dimethoxyphenoxy)-4-{7-[2-(2-methoxyethoxy)ethyl]-3,7-diazabicyclo[3.3.1]non-3-yl}butyl)benzonitrile (m/z=538);

4-[(3-{7-[2-(2-methoxyethoxy)ethyl]-3,7-diazabicyclo[3.3.1]non-3-yl}-propyl)sulfonyl]benzonitrile (m/z=436);

4-cyano-N-(3-{7-[2-(2-methoxyethoxy)ethyl]-3,7-diazabicyclo[3.3.1]non-3-yl}propyl)benzamide (m/z=415);

4-(2-hydroxy-3-{7-[2-(2-methoxyethoxy)ethyl]-3,7-diazabicyclo[3.3.1]-non-3-yl}propoxy)-N,N-dimethylbenzenesulfonamide (m/z=486);

N-{2-[7-(1-cyanoethyl)-3,7-diazabicyclo[3.3.1]non-3-yl]-1-[(4-cyano-phenoxy)methyl]ethyl}-N′-methylurea;

4-cyano-N-{3-[7-(2-cyanopropyl)-3,7-diazabicyclo[3.3.1]non-3-yl]-propyl}benzamide (m/z=380);

N-(2-(4-cyanophenoxy)-1-{[7-(3,4-dimethoxyphenethyl)-3,7-diazabicyclo-[3.3.1]non-3-yl]methyl}ethyl)-N′-methylurea (m/z=522);

4-{3-[7-(3,4-dimethoxyphenethyl)-3,7-diazabicyclo[3.3.1]non-3-yl]-2-hydroxypropoxy}-N,N-dimethylbenzenesulfonamide (m/z=548);

4-{2-[7-(4-oxoheptyl)-3,7-diazabicyclo[3.3.1]non-3-yl]ethoxy}benzonitrile (m/z=384);

4-{2-hydroxy-3-[7-(4-oxoheptyl)-3,7-diazabicyclo[3.3.1]non-3-yl]-propoxy}-N,N-dimethylbenzenesulfonamide (mn/z 496);

N-[2-(4-cyanophenoxy)-1-({7-[2-(2,3-dihydro-1,4-benzodioxin-6-yl)-2-oxoethyl]-3,7-diazabicyclo[3.3.1]non-3-yl}methyl)ethyl]-N′-methylurea (m/z=496);

4-[(3-{7-[2-(2,3-dihydro-1,4-benzodioxin-6-yl)-2-oxoethyl]-3,7-diazabicyclo[3.3.1]non-3-yl}propyl)sulfonyl]benzonitrile (m/z=510);

4-[4-(4-cyanophenyl)-1-(2-{7-[2-(2,3-dihydro-1,4-benzodioxin-6-yl)-2-oxoethyl]-3,7-diazabicyclo[3.3.1]non-3-yl}-2-oxoethyl)-1H-pyrazol-5-yl]-benzonitrile (m/z=510);

4-cyano-N-(3-{7-[2-(2,3-dihydro-1,4-benzodioxin-6-yl)-2-oxoethyl]-3,7-diazabicyclo[3.3.1]non-3-yl}propyl)benzamide (m/z=489);

4-(3-{7-[2-(2,3-dihydro-1,4-benzodioxin-6-yl)-2-oxoethyl]-3,7-diazabicyclo[3.3.1]non-3-yl}-2-hydroxypropoxy)-N,N-dimethylbenzene-sulfonamide (m/z=560);

4-(2-{7-[3-(ethylsulfonyl)propyl]-3,7-diazabicyclo[3.3.1]non-3-yl}ethoxy)-benzonitrile (m/z=406);

N-[2-(4-cyanophenoxy)-1-({7-[3-(ethylsulfonyl)propyl]-3,7-diazabicyclo-[3.3.1]non-3-yl}methyl)ethyl]-N′-methylurea (m/z=492);

4-[(3-{7-[3-(ethylsulfonyl)propyl]-3,7-diazabicyclo[3.3.1]non-3-yl}-propyl)amino]benzonitrile (m/z=419);

4-[(3-{7-[3-(ethylsulfonyl)propyl]-3,7-diazabicyclo[3.3.1]non-3-yl}-propyl)sulfonyl]benzonitrile (m/z=468);

4-[4-(4-cyanophenyl)-1-(2-{7-[3-(ethylsulfonyl)propyl]-3,7-diazabicyclo-[3.3.1]non-3-yl}-2-oxoethyl)-1H-pyrazol-5-yl]benzonitrile (m/z=571);

4-cyano-N-(3-{7-[3-(ethylsulfonyl)propyl]-3,7-diazabicyclo[3.3.1]non-3-yl}propyl)benzamide (m/z=447);

4-(3-{7-[3-(ethylsulfonyl)propyl]-3,7-diazabicyclo[3.3.1]non-3-yl}-2-hydroxypropoxy)-N,N-dimethylbenzenesulfonamide (m/z=518);

4-(2-{7-[3-(4-acetyl-1-piperazinyl)propyl]-3,7-diazabicyclo[3.3.1]non-3-yl}ethoxy)benzonitrile (m/z=440);

4-[4-{7-[3-(4-acetyl 1-piperazinyl)propyl]-3,7-diazabicyclo[3.3.1]non-3-yl}-1-(3,4-dimethoxyphenoxy)butyl]benzonitrile (m/z=604);

4-[(3-{7-[3-(4-acetyl-1-piperazinyl)propyl]-3,7-diazabicyclo[3.3.1]non-3-yl}propyl)amino]benzonitrile (m/z=453);

4-[(3-{7-[3-(4-acetyl-1-piperazinyl)propyl]-3,7-diazabicyclo[3.3.1]non-3-yl}propyl)sulfonyl]benzonitrile (m/z=502);

4-(3-{7-[3-(4-acetyl-1-piperazinyl)propyl]-3,7-diazabicyclo[3.3.1]non-3-yl}-2-hydroxypropoxy)-N,N-dimethylbenzenesulfonamide (m/z=552);

4-(3-{7-[3-(4-acetyl-1-piperazinyl)propyl]-3,7-diazabicyclo[3.3.1]non-3-yl}-2-hydroxypropoxy)benzonitrile;

N-(2-(4-cyanophenoxy)-1-{[7-(1,3-thiazol-2-yl)-3,7-diazabicyclo[3.3.1]-non-3-yl]methyl}ethyl)-N′-methylurea (m/z=441);

4-{1-(3,4-dimethoxyphenoxy)-4-[7-(2-ethyl-2H-1,2,3,4-tetraazol-5-yl)-3,7-diazabicyclo[3.3.1]non-3-yl]butyl}benzonitrile; and

4-({3-[7-(2-ethyl-2H-1,2,3,4-tetraazol-5-yl)-3,7-diazabicyclo[3.3.1]non-3-yl]propyl}amino)benzonitrile.

### EXAMPLE 8

Title compounds of the above Examples were tested in Test A above and were found to exhibit D₁₀ values of at least 6.0.

### EXAMPLE 9

Title compounds of the above Examples were tested in Test B above and were found to exhibit pIC₅₀values of at least 5.5.

Abbreviations

Ac = acetyl
API = atmospheric pressure ionisation (in relation to MS)
aq. = aqueous
br = broad (in relation to NMR)
Bt = benzotriazole
t-BuOH = tert-butanol
CI = chemical ionisation (in relation to MS)
mCPBA = meta-chloroperoxybenzoic acid
d = doublet (in relation to NMR)
DBU = diazabicyclo[5.4.0]undec-7-ene
DCM = dichloromethane
dd = doublet of doublets (in relation to NMR)
DMAP = 4-dimdethylaminopyridine
DMF = N,N-dimethylformamide
DMSO = dimethylsulfoxide
EDC = 1-[3-(dimethylamino)propyl]-3-ethylcarbodiimide
Et = ethyl
EtOAc = ethyl acetate
eq. = equivalents
ES = electrospray (in relation to MS)
FAB = fast atom bombardment (in relation to MS)
h = hour(s)
HCl = hydrochloric acid
HEPES = 4-(2-hydroxyethyl)-1-piperazineethanesulfonic acid
HPLC = high performance liquid chromatography
IPA = iso-propyl alcohol (propan-2-ol)
m = multiplet (in relation to NMR)
Me = methyl
MeCN = acetonitrile
MeOH = methanol
min. = minute(s)
m.p. = melting point
MS = mass spectroscopy
NADPH = nicotinamide adenine dinucleotide phosphate, reduced form
OAc = acetate
Pd/C = palladium on carbon
q = quartet (in relation to NMR)
rt = room temperature
s = singlet (in relation to NMR)
t = triplet (in relation to NMR)
TEA = triethylamine
THF = tetrahydrofuran
tlc = thin layer chromatography
Prefixes n-, s-, i-, t- and tert- have their usual meanings: normal, secondary, iso, and tertiary.

1. A compound of formula I, 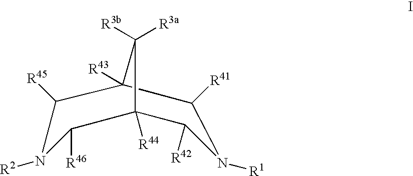
wherein
R¹ represents a structural fragment of formula Ia, 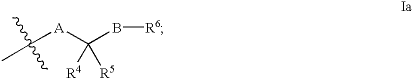
R⁴ represents H, halo, C₁₋₄ alkyl, —D—OR⁷, —D—N(R⁸)R⁹, or R⁴, together with R⁵, represents ═O;
R⁵ represents H, C₁₋₄ alkyl, or R⁵, together with R⁴, represents ═O;
D represents a direct bond or C₁₋₄ alkylene;
R⁷ represents H, C₁₋₆ alkyl, —E—aryl, —E—Het¹, —C(O)R^{l0a}, —C(O)OR^{l0b} or —C(O)N(R^{11a})R^{11b};
R⁸represents H, C₁₋₆ alkyl, —E—aryl, —E—Het¹, —C(O)R^{10a}, —C(O)OR^{10b}, —S(O)₂R^{l0c}, —[C(O)]ₙN(R^{11a})R^{11b} or —C(NH)NH₂;
R⁹represents H, C¹⁻⁶ alkyl, —E—aryl, or —C(O)R^{10d};
E represents, at each occurrence when used herein, a direct bond or C₁₋₄ alkylene;
R^{10a} to R^{10d} independently represent, at each occurrence when used herein, C₁₋₆ alkyl (optionally substituted and/or terminated by one or more substituents selected from halo, aryl and Het²), aryl, Het³, or R^{10a} and R^{10d} independently represent H;
R^{11a} and R^{11b} independently represent, at each occurrence when used herein, H, C₁₋₆ alkyl (optionally substituted and/or terminated by one or more substituents selected from halo, aryl and Het⁴), aryl, Het⁵, or R^{11a} and R^{11b} together represent C₃₋₇ alkylene, which alkylene group is optionally interrupted by an oxygen atom;
n represents 1 or 2;
A represents —G—, —J—N(R¹²)—or —J—O— (in which latter two groups, J is attached to the bispidine nitrogen atom);
B represents —L—, —L—N(R¹³)—, —N(R¹³)—L—, —L—S(O)ₚ— or —L—O— (in which latter two groups, L is attached to the carbon atom bearing R⁴and R⁵);
G represents a direct bond or C₁₋₆ alkylene;
J represents C₂₋₆ alkylene;
L represents a direct bond or C₁₋₄ alkylene;
p represents 0, 1 or 2;
R¹² and R¹³ independently represent H or C₁₋₄ alkyl;
R⁶ represents aryl, Het⁶ (both of which groups are optionally substituted and/or terminated (as appropriate) by one or more substituents selected from —OH, cyano, halo, nitro, C₁₋₆ alkyl (optionally terminated by —N(H)C(O)OR^{14a}), C₁₋₆ alkoxy, aryl, Het⁷, —N(R^{15a})R^{15b}, —C(O)R^{15c}, —C(O)OR^{15d}, —C(O)N(R^{15e})R^{15f}, —N(R^{15g})C(O)R^{15h}, —N(R¹⁵ⁱ)C(O)N(R^{15j})R^{15k}, —N(R^{15m})S(O)₂R^{14b}, —S(O)_{q}R^{14c}, —OS(O)₂R^{14d} and —S(O)₂N(R¹⁵ⁿ)R^{15p}) or, when R⁴ and R⁵ together represent ═O, R⁶ may represent C₁₋₆ alkyl;
q represents 0, 1 or 2;
R² represents —CN, Het⁸, —C(O)R¹⁶, —C(S)OR¹⁷, —C(S)N(R¹⁸)R¹⁹, —[C(O)]₂N(R^{20a})R^{20b}, —[C(O)]₂OR²¹, —S(O)₂R²², —S(O)₂N(R²³)R²⁴, —C(═N—CN)N(R²⁵)R²⁶, —C(═N—CN)OR²⁷ or C₁₋₂ alkyl (which alkyl group is optionally substituted and/or terminated by one or more substituents selected from —C(O)R²⁸, —C(O)N(R^{29a})R^{29b}, —N(R³⁰)R³¹, —OR³², —S(O)ᵣR³³, halo, —CN, nitro, aryl and Het⁹);
R¹⁶ represents H, aryl, Het¹⁰ or C₁₋₆ alkyl (which alkyl group is optionally substituted and/or terminated by one or more substituents selected from halo, —OH, —CN, —N(R³⁴)R³⁵, aryl and Het¹¹);
R³⁴ represents, H, C₁₋₆ alkyl, aryl, Het¹², —C(O)R^{36a} a or —C(O)OR^{36b};
R¹⁸ represents H, aryl, Het¹³, —C(O)R^{36a}, —C(O)OR^{36b} or C₁₋₆ alkyl (which alkyl group is optionally substituted and/or terminated by one or more substituents selected from halo, —OH, —CN, —C(O)R^{36a} and —C(O)OR^{36b});
R²² represents Het¹⁴, aryl, or C₁₋₆ alkyl (which alkyl group is optionally substituted and/or terminated by one or more substituents selected from halo, —OH, —CN, Het¹⁵ and aryl);
R²³ represents H, C₁₋₆ alkyl, aryl, Het¹⁶, —C(O)R^{36a}, —C(O)OR^{36b} or —C(O)SR^{36b};
R²⁵ represents H or C₁₋₆ alkyl (which alkyl group is optionally substituted and/or terminated by one or more substituents selected from halo, —OH, —CN, C₁₋₆ alkyl (which alkyl group is optionally substituted and/or terminated by one or more substituents selected from C₁₋₄ alkyl and —OH), C₁₋₆ alkoxy and aryl);
R²⁷ represents C₁₋₆ alkyl or aryl;
R²⁸represents H, C₁₋₆ alkyl, aryl or Het¹⁷;
R^{29a} and R^{29b} independently represent H, C₁₋₆ alkyl, aryl or Het¹⁸;
R³⁰ represents H, C₁₋₆ alkyl, aryl, Het¹⁹,—C(O)R^{37a}, —C(O)OR^{37b} or —C(O)N(R^{37c})R^{37d};
R³¹ represents H, C¹⁻⁶ alkyl, aryl or Het²⁰;
R³² represents H, C₁₋₆ alkyl, aryl, Het²¹, —C(O)R^{37a}, —C(O)OR^{37b} or —C(O)N(R^{37c})R^{37d};
R³³ represents C₁₋₆ alkyl, aryl or Het²²; r represents 0, 1 or 2;
R^{36a} and R^{36b} independently represent, at each occurrence when used herein, C₁₋₆ alkyl, or R^{36a} represents H;
R^{37a} to R^{37d} independently represent, at each occurrence when used herein, C₁₋₆ alkyl, aryl or Het²³, or R^{37a}, R^{37c} and R^{37d} independently represent H;
Het¹ to Het²³ independently represent, at each occurrence when used herein, five- to twelve-membered heterocyclic groups containing one or more heteroatoms selected from oxygen, nitrogen and/or sulfur;
R^{3a} and R^{3b} independently represent H, C₁₄ alkyl, -OR^{38a}, —SR^{38b},
—N(R³⁹)R^{38c}, or R^{3a} and R^{3b} together represent C₃₋₅ alkylene, —O—Z—O—, —O—Z—S— or —S—Z—S—;
R³⁹ represents H, C₁₋₆ alkyl or a structural fragment of formula Ia as defined above;
Z represents C₂₋₃ alkylene optionally substituted by one or more C₁₋₄ alkyl groups;
R⁴¹ to R⁴⁶ independently represent H or C₁₋₃ alkyl; R^{14a} to R^{14d}, R¹⁷ and R²¹ independently represent C₁₋₆ alkyl;
R^{15a} to R^{15p}, R¹⁹, R^{20a}, R^{20b}, R²⁴, R²⁶, R³⁵ and R^{38a} to R^{38c} independently represent H or C₁₋₆ alkyl;
wherein each aryl and Het (Het¹ to Het²³) group, unless otherwise specified, is optionally substituted;
or a pharmaceutically acceptable derivative thereof;
provided that:
(a) when R¹ represents a structural fragment of formula Ia in which:
R⁴ and R⁵ together represent ═O;
A represents a direct bond;
then B does not represent a direct bond, —N(R¹³)—L— (in which group —N(R¹³)—is attached to the carbon atom bearing R⁴ and R⁵), —N(R¹³)—, —S(O)ₚ— or —O—;
(b) when R⁵ represents H or C₁₋₄ alkyl; and
A represents —J—N(R¹²)— or —J—O—,
then B does not represent —N(R¹³)—L—, —N(R¹³)—, —S(O)ₚ— or —O—;
(c) when R⁴ represents —D—OR⁷, —D—N(R⁸)R⁹ in which D represents a direct bond, then:
(i) A does not represent —J—N(R¹²)— or —J—O—; and
(ii) B does not represent —N(R¹³)—L—, —N(R¹³)—, —S(O)ₚ— or —O—;
(d) when R^{3a} and R^{3b} and both represent H;
and R¹ represents unsubstituted benzyl;
then R² does not represent unsubstituted benzyl or optionally substituted benzoyl; and
(e) the compound is not:
(i) N¹-phenyl-3-(7-benzyl-3,7-diazabicyclo[3.3.1]non-3-yl)-propanamide;
(ii) 3-benzyl-7-[3-(4-cyanophenoxy)-2-hydroxypropyl]-6,8-dimethyl-3,7-diazabicyclo[3.3.1]nonane;
(iii) 3-benzyl-7-[3-(4-cyanophenoxy)-2-hydroxypropyl]-6-methyl-3,7-diazabicyclo[3.3.1]nonane;
(iv) N-{2-(7-benzyl-3,7-diazabicyclo[3.3.1]non-3-yl)-1-[(4-cyanophenoxy)methyl]ethyl}methanesulfonamide;
(v) 3-benzyl-7-[3-(2-propyl-1,3-dioxolan-2-yl)propyl]-3,7-diazabicyclo[3.3.1]nonane; or
(vi) 7-benzyl-3,7-diazabicyclo[3.3.1]nonane-3-ethanol.

2. A compound as claimed in claim 1, wherein R⁴ represents H, C₁₋₂ alkyl, —OR⁷ or N(H)R⁸, or R⁴, together with R⁵, represents ═O.

3. A compound as claimed in claim 1 or claim 2, wherein R⁵ represents H, or R⁵, together with R⁴, represents ═O.

4. A compound as claimed in any one of claims 1 to 3, wherein R⁷ represents H, C₁₋₄ alkyl, optionally substituted phenyl, —C(O)R^{10a}, or —C(O)N(R^{11a})R^{11b}.

5. A compound as claimed in any one of claims 1 to 3, wherein R⁸ represents H, C₁₋₄ alkyl, —C(O)R^{10a}, —C(O)OR^{10b} or —C(O)N(R^{11a})R^{11b}.

6. A compound as claimed in any one of claims 1 to 5, wherein R^{10a} and R^{10b} independently represent, at each occurrence when used herein, C₁₋₅ alkyl (optionally substituted and/or terminated by one or more substituents selected from halo and phenyl), optionally substituted phenyl, or R^{10a} represents H.

7. A compound as claimed in claim 4 or claim 5, wherein R^{11a} and R^{11b} independently represent, at each occurrence when used herein, H or C₁₋₅ alkyl (optionally substituted and/or terminated by one or more substituents selected from halo and phenyl).

8. A compound as claimed in any one of claims 1 to 7, wherein A represents —G—or —J—N(R¹²)—.

9. A compound as claimed in claim 8, wherein G represents a direct to bond or C₁₋₄ alkylene.

10. A compound as claimed in any one of claims 1 to 8, wherein J represents C₂₋₄ alkylene.

11. A compound as claimed in any one of claims 1 to 10, wherein B represents a direct bond, C₁₋₄ alkylene, —L—N(H)—, —L—S(O)₂— or —L—O— (in which latter three groups, L is attached to the carbon atom bearing R⁴and R⁵).

12. A compound as claimed in any one of claims 1 to 11, wherein L represents C₁₋₄ alkylene.

13. A compound as claimed in any one of claims 1 to 12, wherein R⁶ represents phenyl, Het⁶ (both of which groups are optionally substituted by one or more substituents selected from cyano, halo, nitro, C₁₋₄ alkyl, C₁₋₄ alkoxy, optionally substituted phenyl, —N(H)R^{15b}, 13 C(O)R^{15c}, —C(O)N(H)R^{15f}, —N(H)C(O)RI^{15h}, —N(H)C(O)N(H)R^{15k}, —N(H)S(O)₂R^{14b}, —S(O)₂R^{14c} and —S(O)₂N(R¹⁵ⁿ)R^{15p}), or, when R⁴ and R⁵ together represent ═O, R⁶may represent C₁₋₅ alkyl.

14. A compound as claimed in any one of claims 1 to 13, wherein R² represents —CN, Het⁸, —C(O)R¹⁶, —C(S)OR¹⁷, —C(S)N(H)R¹⁸, —[C(O)]₂N(H)R^{20b}, —[C(O)]₂OR²¹, —S(O)₂R²², —S(O)₂N(R²³)R²⁴, —C(═—N—CN)N(R²⁵)R²⁶, —C(═N—CN)OR²⁷ or C₁₋₆ alkyl (which alkyl group is optionally substituted and/or terminated by one or more substituents selected from —C(O)R²⁸, —C(O)N(H)R^{29b}, —N(R³⁰)R³¹, —OR³², —S(O)₂R³³, halo, —CN, optionally substituted phenyl and Het⁹).

15. A compound as claimed in claim 14, wherein R¹⁶ represents optionally substituted phenyl, Het¹⁰ or C₁₋₆ alkyl (which alkyl group is optionally unsaturated and/or optionally substituted and/or terminated by one or more substituents selected from halo, —CN, —N(H)R³⁴ and optionally substituted phenyl).

16. A compound as claimed in claim 15, wherein R³⁴ represents, H, C₁₋₄ alkyl, —C(O)R^{36a} or —C(O)OR^{36a}.

17. A compound as claimed in claim 14, wherein R¹⁸ represents H, —C(O)OR^{36b} or C₁₋₆ alkyl (which alkyl group is optionally substituted and/or terminated by one or more substituents selected from halo and —C(O)OR^{36b}).

18. A compound as claimed in claim 14, wherein R²² represents Het¹⁴, optionally substituted phenyl or C₁₋₄ alkyl (which alkyl group is optionally substituted and/or terminated by one or more substituents selected from halo, Het¹⁵ and optionally substituted phenyl).

19. A compound as claimed in claim 14, wherein R²³ represents H, C₁₋₄ alkyl, —C(O)OR^{36b} or —C(O)SR^{36b}.

20. A compound as claimed in claim 14, wherein R²⁵ represents H or C₁₋₆ alkyl (which alkyl group is optionally substituted and/or terminated by one or more substituents selected from halo, —OH, C₁₋₆ alkyl (which alkyl group is optionally substituted and/or terminated by one or more substituents selected from C₁₋₄ alkyl and —OH), C₁₋₄ alkoxy, naphthyl and optionally substituted phenyl).

21. A compound as claimed in claim 14, wherein R²⁷ represents optionally substituted phenyl.

22. A compound as claimed in claim 14, wherein R²⁸ represents C₁₋₅ alkyl, optionally substituted phenyl or Het¹⁷.

23. A compound as claimed in any one of claims 1 to 14 or 22, wherein R^{29b} represents H, C₁₋₄ alkyl or optionally substituted phenyl.

24. A compound as claimed in any one of claims 14, 22 and 23, wherein R³⁰ represents H, optionally substituted phenyl, —C(O)R^{37a} or —C(O)OR^{37b}.

25. A compound as claimed in any one of claims 14 or 22 to 24, wherein R³¹ represents H, C₁₋₂ alkyl or optionally substituted phenyl.

26. A compound as claimed in any one of claims 14 or 22 to 25, wherein R³² represents H, C₁₋₄ alkyl (which alkyl group is optionally interrupted by oxygen), optionally substituted phenyl or Het²¹.

27. A compound as claimed in any one of claims 14 or 22 to 26, wherein R³³ represents C₁₋₆ alkyl or optionally substituted phenyl.

28. A compound as claimed in any one of claims 1 to 14 or 22 to 27, wherein R^{37a} and R^{37b} independently represent, at each occurrence when used herein, C₁₋₅ alky, optionally substituted phenyl, or R^{37a} represents H.

29. A compound as claimed in any one of claims 1 to 28, wherein R^{3a} and R^{3b} independently represent H, C₁₋₂ alkyl, —SR^{38b}, —N(R³⁹)R^{38c}, or R^{3a} and R^{3b} together represent C₃₋₄ alkylene or —O—Z—O—.

30. A compound as claimed in claim 29, wherein R³⁹ represents H, C₁₋₂ alkyl or a structural fragment of formula Ia.

31. A compound as claimed in any one of claims 1 to 30, wherein Z represents C₂₋₃ alkylene.

32. A compound as claimed in any one of claims 1 to 31, wherein R⁴¹ to R⁴⁶ independently represent H or C₁₋₂ alkyl.

33. A compound as claimed in claim 13 or claim 14, wherein R^{14b}, R^{14c}, R¹⁷ and R²¹ independently represent C₁₋₄ alkyl.

34. A compound as claimed in any one of claims 13, 14 and 29, wherein R^{15b} to R^{15p}, R^{20b}, R²⁴,R²⁶, R^{38b} and R^{38c} independently represent H or C₁₋₅ alkyl.

35. A compound as claimed in any one of claims 1 to 34, wherein optional substituents on phenyl groups are one or more substituents selected from cyano, halo, nitro, C₁₋₂ alkyl, C₁₋₂ alkoxy, Het¹, —NH₂, —C(O)R^{15c}, —C(O)N(H)R^{15f}, —N(H)C(O)R^{15h}, —N(H)C(O)N(H)R^{15k}, —N(H)S(O)₂R^{14b} and —S(O)₂N(R¹⁵ⁿ)R^{15p}.

36. A pharmaceutical formulation including a compound as defined in any one of claims 1 to 35 in admixture with a pharmaceutically-acceptable adjuvant, diluent or carrier.

37. A pharmaceutical formulation for use in the prophylaxis or the treatment of an arrhythmia, comprising a compound as defined in any one of claims 1 to 35.

38. A compound as defined in any one of claims 1 to 35, but without proviso (e), for use as a pharmaceutical.

39. A compound as defined in any one of claims 1 to 35, but without proviso (e), for use in the prophylaxis or the treatment of an arrhythmia.

40. The use of a compound as defined in any of one claims 1 to 35, but without proviso (e), as active ingredient for the manufacture of a medicament for use in the prophylaxis or the treatment of an arrhythmia.

41. The use as claimed in claim 40, wherein the arrhythmia is an atrial or a ventricular arrhythmia.

42. A method of prophylaxis or treatment of an arrhythmia which method comprises administration of a therapeutically effective amount of a compound as defined in any one of claims 1 to 35, but without proviso (e), to a person suffering from, or susceptible to, such a condition.

43. A process for the preparation of a compound of formula I as defined in claim 1 which comprises:
(a) reaction of a corresponding compound of formula II, 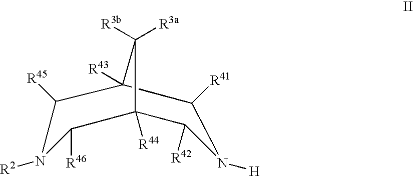
wherein R², R^{3a}, R^{3b} and R⁴¹ to R⁴⁶ are as defined in claim 1, with a compound of formula III, 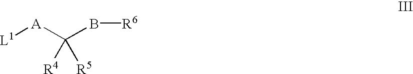
wherein L¹ represents a leaving group and R⁴, R⁵, R⁶, A and B are as defined in claim 1;
(b) for compounds of formula I in which R¹ represents a structural fragment of formula Ia in which A represents C₂ alkylene and R⁴ and R⁵ together represent ═O, reaction of a corresponding compound of formula II, as defined above, with a compound of formula IV, 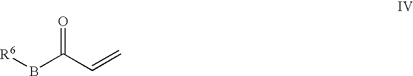
wherein R⁶ and B are as defined in claim 1;
(c) for compounds of formula I in which R^{3a} or R^{3b} represents —N(R³⁹)R^{38c} and R³⁹ represents a structural fragment of formula Ia, reaction of a corresponding compound of formula I in which R^{3a} or R^{3b} (as appropriate). represents —N(H)R^{38c}, wherein R^{38c} is as defined in claim 1, with a compound of formula III as defined above;
(d) for compounds of formula I in which R¹ represents a fragment of formula Ia in which A represents CH₂ and R⁴ represents —OH or —N(H)R⁸, reaction of a corresponding compound of formula II, as defined above, with a compound of formula V, 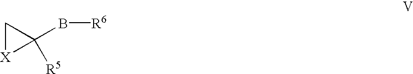
wherein X represents O or N(R⁸) and R⁵, R⁶, R⁸ and B are as defined in claim 1;
(e) for compounds of formula I in which R^{3a} or R^{3b} represents —N(R³⁹)R^{38c} and R³⁹ represents a structural fragment of formula Ia in which A represents CH₂ and R⁴ represents —OH or —N(H)R⁸, reaction of a corresponding compound of formula I in which R^{3a} or R^{3b} (as appropriate) represents —N(H)R^{38c}, wherein R^{38c} is as defined in claim 1, with a compound of formula V as defined above;
(f) for compounds of formula I in which A represents C₁₋₆ alkylene, B represents C₁₋₄ alkylene and R⁴ and R⁵ both represent H, reduction of a corresponding compound of formula I in which R⁴ and R⁵ together represent ═O;
(g) for compounds of formula I in which R⁴ and R⁵ both represent H and (1) A represents a single bond or —J—N(R¹²) and B represents C₁₋₄ alkylene, or (2) A represents C₁₋₆alkylene and B represents N(R¹³) or —N(R ¹³)—L—, reduction of a corresponding compound of formula I in which R⁴ and R⁵ together represent ═O;
(h) for compounds of formula I in which A represents C₁₋₆ alkylene, B represents a direct bond, C₁₋₄ alkylene, —L—N(R¹⁻³)—, —L—S(O)ₚ— or —L—O— (in which latter three groups L represents C₁₋₄ alkylene), R⁴ represents OH and R⁵ represents H, reduction of a corresponding compound of formula I in which R⁴ and R⁵ together represent ═O;
(i) for compounds of formula I in which R^{3a} and R^{3b} both represent H, reduction of a corresponding compound of formula VI, 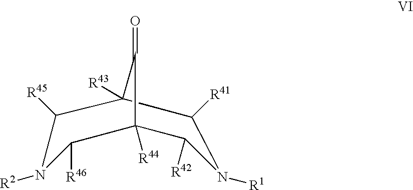
wherein R¹, R² and R⁴¹ to R⁴⁶ are as defined in claim 1, and in which the bridgehead C═O group may be activated;
(j) for compounds of formula I in which one of R^{3a} and R^{3b} represents H, and the other represents —OH, reduction of a corresponding compound of formula VI, as defined above;
(k) for compounds of formula I in which R^{3a} and R^{3b} both represent —OR^{38a} or —SR^{38b}, or in which R^{3a} and R^{3b} together represent —O—Z—O—, —O—Z—S— or —S—Z—S—, reaction of a corresponding compound of formula VI, as defined lo above, with a compound of formula HOR^{38a}, HSR^{38b}, HO—Z—OH, HO—Z—SH or HS—Z—SH (as appropriate), wherein R^{38a}, R^{38b} and Z are as defined in claim 1;
(I) for compounds of formula I in which one of R^{3a} and R^{3b} represents —NH₂ and the other represents H, reduction of a compound of formula VII, 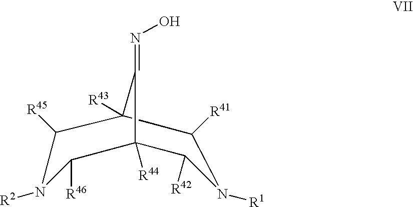
wherein R¹, R² and R⁴¹ to ⁴⁶ are as defined in claim 1;
(m) for compounds of formula I in which one or both of R^{3a} and R^{3b} represent —N(R³⁹)R^{38c} in which one or both of R³⁹ and R^{38c} represents C₁₋₆ alkyl, alkylation of a corresponding compound of formula I in which R^{3a} and/or R^{3b} represent —N(R³⁹)R^{38c} (as appropriate) in which R³⁹ and/or R^{38c} (as appropriate) represent H, using a compound of formula VIII,
R^{a}—L¹ VIII
wherein R^{a} represents C₁₋₆ alkyl and L¹ is as defined above;
(n) for compounds of formula I in which R¹ represents a structural fragment of formula Ia in which B represents —L—O—, reaction of a compound of formula TX, 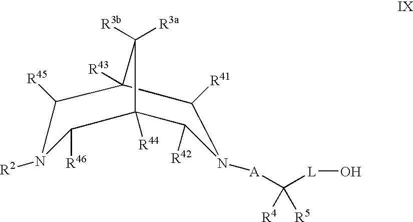
wherein R², R^{3a}, R^{3b}, R⁴, R⁵, R⁴¹ to R⁴⁶, A and L are as defined in claim 1, with a compound of formula X,
R⁶OH X
in which R⁶ is as defined in claim 1;
(o) for compounds of formula I in which R¹ represents a structural fragment of formula Ia in which A represents C₁₋₆ alkylene and B represents —N(R¹³)—L— (wherein the group —N(R¹³)— is attached to the carbon atom bearing R⁴ and R⁵), reaction of a compound of formula XI, 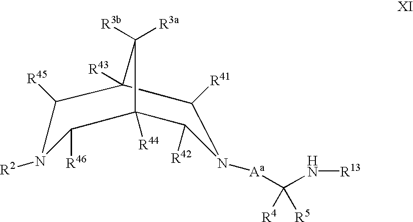
wherein Aa represents C1-6 alkylene and R², R^{3a}, R^{3b}, R⁴, R⁵, R¹³ and R⁴¹ to R⁴⁶ are as defined in claim 1, with a compound of formula XII,
R⁶—L—L² XII
wherein L represents a leaving group and R⁶ and L are as defined in claim 1;
(p) for compounds of formula I in which R¹ represents a structural fragment of formula Ia in which R⁴ represents —D—NH₂, reduction of a corresponding compound of formula XIII, 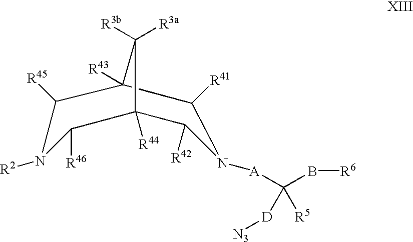
wherein R², R^{3a}, R^{3b}, R⁵,R⁶, R⁴¹ to R⁴⁶, A, B and D are as defined in Claim 1;
(q) for compounds of formula I in which R⁴ represents —D—N(R⁹)C(O)NH(R^{11b}), reaction of a corresponding compound of formula I in which R⁴ represents —D—N(R⁹)H with a compound of formula XIV,
R^{11b}N═C═O XIV
wherein R^{11b} is as defined in claim 1;
(r) for compounds of formula I in which R⁴ represents —D—N(H)[C(O)]₂NH₂, reaction of a corresponding compound of formula I in which R⁴ represents —D—NH₂ with oxalic acid diamide;
(s) for compounds of formula I in which R⁴ represents —D—N(R⁸)R⁹, wherein R⁸ and R⁹ are as defined in claim 1, provided that R⁸ does not represent H, reaction of a corresponding compound of formula I, in which R⁴ represents —D—N(H)R⁹ with a compound of formula XV,
R^{8a}—L³ XV
wherein R^{8a} represents R⁸ as defined in claim 1 except that it does not represent H, and L³ represents a leaving group;
(t) for compounds of formula I in which R⁴ represents —D—OR⁷ in which R⁷ represents C₁₋₆ alkyl, —E—aryl or —E—Het¹, reaction of a corresponding compound of formula I in which R⁴ represents —D—OH with a compound of formula XVI,
R^{7a}OH XVI
wherein R^{7a} represents C₁₋₆ alkyl, —E—aryl or —E—Het¹, wherein Het¹ is as defined in claim 1;
(u) for compounds of formula I in which R¹ represents a structural fragment of formula la in which R⁴ represents —D—OR⁷ (in which R⁷ represents C₁₋₆ alkyl, —E—aryl or —E—Het¹), reaction of a corresponding compound of formula XVII, 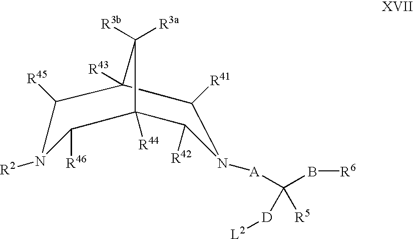
wherein R²,R^{3a}, R^{3b}, R⁵, R⁶, R⁴¹ to R⁴⁶, A, B and D are as defined in claim 1 and L² is as defined above, with a compound of formula XVI as defined above;
(v) for compounds of formula I in which R⁴ represents —D—OR⁷, wherein R⁷ is as defined in claim 1, provided that it does not represent H, reaction of a corresponding compound of formula I in which R⁴ represents —D—OH with a compound of formula XVIII,
R^{7b}—L⁴ XVIII
wherein R^{7b} represents R⁷ as defined in claim 1, except that it does not represent H, and L⁴ represents a leaving group;
(w) for compounds of formula I in which R⁴ represents halo, substitution of a corresponding compound of formula I in which R⁴ represents —OH, using an appropriate halogenating agent;
(x) reaction of a corresponding compound of formula XIX, 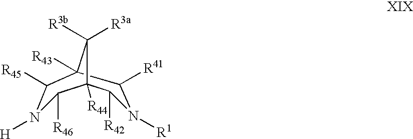
wherein R¹, R^{3a}, R^{3b} and R⁴¹ to R⁴⁶ are as defined in claim 1, with a compound of formula XX,
R²—L⁵ XX
wherein L⁵ represents a leaving group and R² is as defined in claim 1;
(y) for compounds of formula I in which R² represent C₁₋₁₂ alkyl, which alkyl group is substituted at the C-2 carbon (relative to the bispidine nitrogen) with OH or N(H)R³⁰, and is otherwise optionally substituted with one or more further substituents as specified in claim 1 for R², reaction of a compound of formula XIX as defined above with a compound of formula XXA 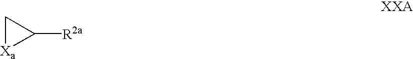
wherein Xa represents O or N(R³⁰) and R^{2a} represents C₁₋₁₀ alkyl, optionally substituted with one or more substituents as specified in claim 1 for R²;
(z) for compounds of formula I in which R² represents tetrazol-5-yl, reaction of a corresponding compound of formula I in which R² represents —CN with a source of the azide ion;
(aa) for compounds of formula I which are bispidine-nitrogen N-oxide derivatives, oxidation of the corresponding bispidine nitrogen of a corresponding compound of formula I, in the presence of a suitable oxidising agent;
(ab) for compounds of formula I which are C₁₋₄ alkyl quaternary ammonium salt derivatives, in which the alkyl group is attached to a bispidine nitrogen, reaction, at the bispidine nitrogen, of a corresponding compound of formula I with a compound of formula XXI,
R^{b}—L² XXI
wherein R^{b} represents C₁₋₄ alkyl and L² is as defined above;
(ac) conversion of one substituent on R⁶ to another;
(ad) conversion of one R² group to another; or
(ae) deprotection of a protected derivative of a compound of formula I as defined in claim 1.

44. A compound of formula II, as defined in claim 43, or a protected derivative thereof.

45. A compound of formula VI, as defined in claim 43, or a protected derivative thereof.

46. A compound of formula VII, as defined in claim 43, or a protected derivative thereof.

47. A compound of formula IX, as defined in claim 43, or a protected derivative thereof.

48. A compound of formula XI, as defined in claim 43, or a protected derivative thereof.

49. A compound of formula XIII, as defined in claim 43, or a protected derivative thereof.

50. A compound of formula XVII, as defined in claim 43, or a protected derivative thereof.

51. A compound of formula XIX, as defined in claim 43 (provided that at least one of R^{3a} and R^{3b} represents —N(R³⁹)R^{38c}, wherein R³⁹ represents a structural fragment of formula Ia, as defined in claim 1), or a protected derivative thereof.

52. A compound of formula XXII, 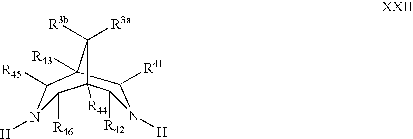
wherein R^{3a}, R^{3b} and R⁴¹ to R⁴⁶ are as defined in claim 1 (provided that at least one of R^{3a} and R^{3b} represents —N(R³⁹)R^{38c}, wherein R³⁹ represents a structural fragment of formula Ia, as defined in claim 1), or a protected derivative thereof.

53. A compound of formula XXIII, 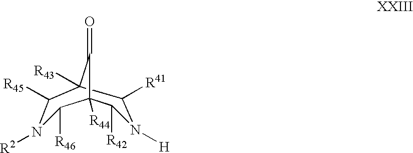
wherein R²and R⁴¹ to R⁴⁶ are as defined in claim 1, or a protected derivative thereof.
